(19) United States
(12) Patent Application Publication
(10) Pub. No.: US 2010/0260698 A1
(43) Pub. Date: Oct. 14, 2010

(54) Anhydrous Antiperspirant Compositions with Improved Active Substance Release

(76) Inventors: Cheryl Galante, Scottsdale, AZ (US); Bernhard Banowski, Dusseldorf (DE); Nadine Buse, Hilden (DE)

Correspondence Address: Henkel Corporation, 10 Finderne Avenue, Suite B, Bridgewater, NJ, 08807, US

(21) Appl. No.: 12/823,484

(22) Filed: Jun. 25, 2010

Related U.S. Application Data
(63) Continuation of application No. PCT/IB2008/003974, filed on Dec. 28, 2008.

(30) Foreign Application Priority Data
Dec. 28, 2007 (DE) 102007063352.3

(51) Int. Cl. A61K 8/92; A61Q 15/00

(52) U.S. Cl. 424/65

(57) ABSTRACT

Antiperspirant compositions for personal body care, ready-made as a stick, soft solid, cream, gel, suspension, solution or impregnated on a substrate, containing at least one antiperspirant active substance, at least one oil, liquid under normal conditions, as a carrier, 0-3 wt. % by weight of free water, based on weight of the composition, and at least one organosiloxane oxyalkylene copolymer.

### CROSS REFERENCE TO RELATED APPLICATIONS

The present application is a continuation of International Application No. PCT/IB2008/003974 filed 22 Dec. 2008, which claims priority to German Patent Application No. 10 2007 063 352.3 filed 28 Dec. 2007.

Anhydrous antiperspirant compositions which allow better release of active substances of the antiperspirant are the object of the present invention.

Anhydrous antiperspirant-compositions, ready-made as a non-aerosol, a stick, a cream, a gel, a suspension or a solution, contain in addition to the antiperspirant active substances, generally at least one cosmetic oil as a carrier for the particulate antiperspirant active substance. In order that the antiperspirant active substance suspended in the oil does not precipitate during storage, commercial suspensions, gels, creams and sticks contain a suspension or thickener agent, for gels, for example hydrophobically modified hectorites, as they are available for example under the commercial designation of Bentone gels from the Rheox and Elementis Specialties Corporations, for sticks, thickeners such as fatty alcohols and/or waxes.

For commercial anhydrous antiperspirant compositions, the antiperspirant active substance suspended in the anhydrous oil-containing carrier is covered with an oil layer. This oil layer certainly delays the release of antiperspirant active substance in the effective water-soluble form.

The object of the present invention was therefore to prepare anhydrous antiperspirant compositions which have improved release of the antiperspirant active substance and so better antiperspirant performance.

Surprisingly, it was now found that the release of the antiperspirant active substance from an anhydrous antiperspirant composition may be improved if at least one organosiloxane-oxyalkylene copolymer is contained.

The object of the present invention accordingly is an antiperspirant composition for personal body care, ready-made as a non-aerosol, a stick, cream, gel, suspension, solution or impregnated on a substrate, containing at least one antiperspirant active substance, at least one oil liquid under normal conditions as a carrier, 0-3% by weight, preferably 0-2% by weight of free water, based on the weight of the composition, and at least one organosiloxane-oxyalkylene copolymer.

Antiperspirant compositions according to the invention on an anhydrous basis are ready-made as a stick, soft solid, cream, gel, suspension, solution or as a substrate impregnated with the composition (tissue, pad, wad, etc.).

For the present application, “normal conditions” refer to a temperature of 20° C. and a pressure of 1013.25 mbar. Indications of melting point also refer to a pressure of 1013.25 mbar.

Preferred compositions according to the invention include an organosiloxane-oxyalkylene copolymer of general structural formulae (I), (II), (III), (IV) and (V)

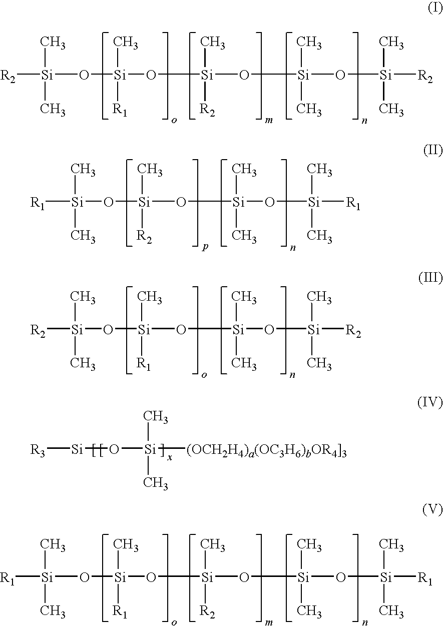

wherein
R¹ is a linear or branched C₁-C₃₀ alkyl group or an optionally substituted phenyl group, preferably a methyl group,
R² is —C_{c}H_{2c}—O—(C₂H₄O—)ₐ(C₃H₆O—)_{b}R⁵ or —CH₂—CH(CH₃)—CH₂—O—(C₂H₄O—)ₐ(C₃H₆O—)_{b}R⁵,
R³ and R⁴ are each independently a linear or branched C₁-C₁₆ alkyl group, preferably methyl groups,
R⁵ is a hydrogen atom or a methyl group,
m is a number from 0-20,
n is a number from 0-500, preferably 20-400, particularly preferably 50-300,
o is a number from 0-20,
p is a number from 1-50, preferably 10-40, particularly preferably 20-30,
a is a number from 0-50, preferably 5 -25, particularly preferably 7-22,
b is a number from 0-50, preferably 0 or 5-30, particularly preferably either 0 or 10 -25, most preferably either 0 or 24,
a+b has the value of at least 1,
c is a number from 1-4, particularly preferably 3, and
x is a number from 1-100.

According to the embodiments of the present invention (composition, use, methods), organosiloxane-oxyalkylene copolymers of general structural formulae (I), (II), (III) and (V), illustrated above, are preferred, wherein R¹ is a linear or branched C₁-C₃₀ alkyl group, preferably a linear or branched C₁-C₁₆ alkyl group, particularly preferably a linear or branched C₁-C₄ alkyl group, most preferably a methyl group. More preferred linear or branched C₁-C₄ alkyl groups include methyl, ethyl, 1-methylethyl, n-propyl, n-butyl, tert-butyl and 2-methylpropyl.

According to the various embodiments of the invention (composition, use, methods), organosiloxane-oxyalkylene copolymers of general structural formula (IV) illustrated above are further preferred, wherein R³ and R⁴ are each independently a linear or branched C₁-C₁₆ alkyl group, preferably a linear or branched C₁-C₆ alkyl group, particularly preferably a linear or branched C₁-C₄ alkyl group, most preferably a methyl group. More preferred linear or branched C₁-C₄ alkyl groups include methyl, ethyl, 1-methylethyl, n-propyl, n-butyl, tert-butyl and 2-methylpropyl.

Further preferred compositions according to the invention include an organosiloxane-oxyalkylene copolymer d) of general structural formulae (I), (II), (III), (IV) and (V) illustrated above having a HLB value in the range of from 8-20, preferably 10-18, particularly preferably 11-16.

Further preferred compositions according to the invention include an organosiloxane-oxyalkylene copolymer d) of general structural formulae (I), (II), (III) and (V), illustrated above, wherein R¹ is a linear or branched C₁-C₃₀ alkyl group, preferably a linear or branched C₁-C₁₆ alkyl group, particularly preferably a linear or branched C₁-C₄ alkyl group, in particular a methyl, ethyl, 1-methylethyl, n-propyl, n-butyl, tert-butyl and 2-methylpropyl, most preferably a methyl group, and have an HLB value in the range from 8-20, preferably 10-18, particularly preferably 11-16.

Further preferred compositions according to the invention include an organosiloxane-oxyalkylene copolymer d) of general structural formula (V), illustrated above, wherein R³ and R⁴ are each independently a linear or branched C₁-C₁₆ alkyl group, preferably a linear or branched C₁-C₆ alkyl group, particularly preferably a linear or branched C₁-C₄ alkyl group, in particular a methyl, ethyl, 1-methylethyl, n-propyl, n-butyl, tert-butyl and 2-methylpropyl, most preferably a methyl group, and have an HLB value in the range from 8-20, preferably 10-18, particularly preferably 11-16.

According to the invention more preferred compositions include an organosiloxane-oxyalkylene copolymer of general formula (III) having an HLB value in the range from 8-20, preferably 10-18, particularly preferably 11-16, and with
R¹=methyl group,
R²═—C_{c}H_{2c}—O—(C₂H₄O—)ₐR⁵,
R⁵=a hydrogen atom or a methyl group,
n=0,
p=1,
a=5-20, preferably 7-15, particularly preferably 8-11,
b=0, and
c=3.

Further more preferred compositions according to the invention include an organosiloxane-oxyalkylene copolymer of general formula (II) having an I-1L13 value in the range from 8-20, preferably 10-18, particularly preferably 11-16, and with
R¹=methyl group,
R²=—C_{c}H_{2c}—O—(C₂H₄O—)ₐR⁵,
R⁵=a hydrogen atom,
n=0,
p=1,
b=0,
a=7, 8, 9, 10, 11, 12, 13, 14 or 15, and
c=3,
preferably chosen from

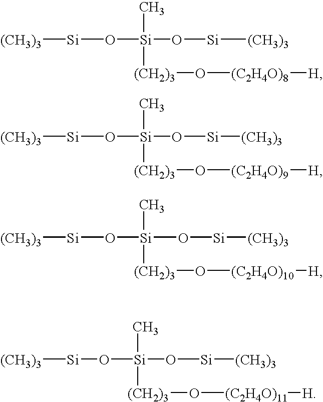

Further more preferred compositions according to the invention include an organosiloxane-oxyalkylene copolymer of general structural formula (II) having an HLB value in the range from 8-20, preferably 10-18, particularly preferably 11-16, and with
R¹=tert-butyl group,
R²═—C_{c}H_{2c}—O—(C₂H₄O—)ₐR⁵,
R⁵=a hydrogen atom or a methyl group,
n=0,
p=1,
a=5-20, preferably 7-15, particularly preferably 8-11,
b=0, and
c=3,
preferably chosen from

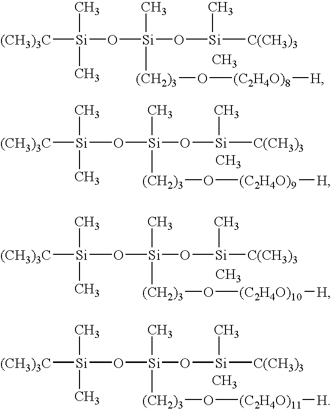

Further more preferred compositions according to the invention include an organosiloxane-oxyalkylene copolymer of general structural formula (II) having an HLB value in the range from 8-20, preferably 10-18, particularly preferably 11-16, and with
R¹=tert-butyl groups,
R²═—C_{c}H_{2c}—O—(C₂H₄O—)ₐR⁵,
R⁵=a hydrogen atom,
n=0,
p=1,
a=7, 8, 9, 10, 11, 12, 13, 14 or 15, and
c=3.

Further more preferred compositions according to the invention include an organosiloxane-oxyalkylene copolymer of general structural formula (II) having an HLB value in the range from 8-20, preferably 10-18, particularly preferably 11-16, and with
R¹=isopropyl groups (—CH(CH₃)₂),
R²═—C_{c}H_{2c}—O—(C₂H₄O—)ₐR⁵,
R⁵=a hydrogen atom or a methyl group,
n=0,
p=1,
a=5-20, preferably 7-15, particularly preferably 8-11, and
c=3.

Further more preferred compositions according to the invention include an organosiloxane-oxyalkylene copolymer of general structural formula (II) having an I-ILB value in the range from 8-20, preferably 10-18, particularly preferably 11-16, and with
R¹=isopropyl groups (—CH(CH₃)₂),
R²═—C_{c}H_{2c}—O—(C₂H₄O—)ₐR⁵,
R⁵=a hydrogen atom,
n=0,
p=1,
a=7, 8, 9, 10, 11, 12, 13, 14 or 15, and
c=3,
preferably chosen from

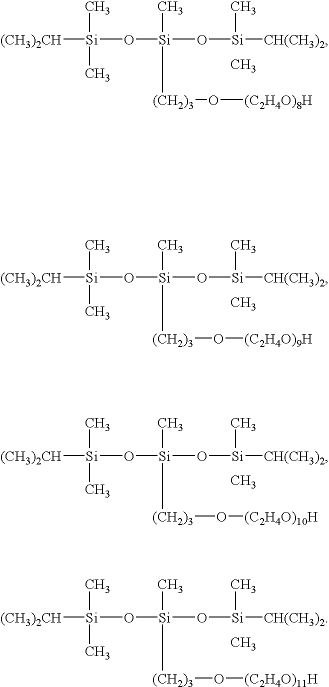

Further more preferred compositions according to the invention include an organosiloxane-oxyalkylene copolymer of general structural formula (II) having an HLB value in the range of from 8-20, preferably 10-18, particularly preferably 11-16, and with
R¹=methyl,
R²═—CH₂—CH(CH₃)—CH₂—O—(C₂H₄O—)ₐR⁵,
R⁵=a hydrogen atom or a methyl group,
n=0,
p=1,
a=5-20, preferably 7-15, particularly preferably 8-11, and
c=3.

Further more preferred compositions according to the invention include an organosiloxane-oxyalkylene copolymer of general structural formula (II) having an HLB value in the range of from 8-20, preferably 10-18, particularly preferably 11-16, and with
R¹=methyl,
R²═—CH₂—CH(CH₃)—CH₂—O—(C₂H₄O—)ₐR⁵,
R⁵=a hydrogen atom,
n=0,
p=1,
a=7, 8, 9, 10, 11, 12, 13, 14 or 15,
c=3,
preferably chosen from

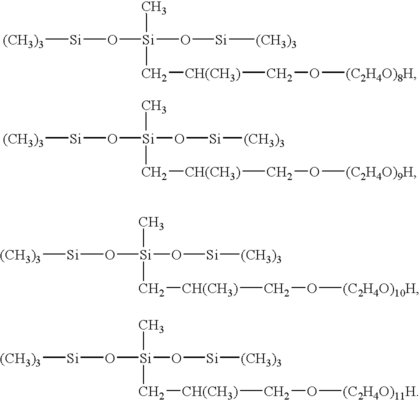

Further more preferred compositions according to the invention include an organosiloxane-oxyalkylene copolymer of general structural formula (II) having an HLB value in the range of from 8-20, preferably 10-18, particularly preferably 11-16, and with
R¹=methyl,
R²═—C_{c}H_{2c}—O—(C₂H₄O—)ₐ(C₃H₆O—)_{b}R⁵,
R⁵=a hydrogen atom or a methyl group,
n=10-500, preferably 20-400, particularly preferably 50-300,
p=10-50,
a=5-30, preferably 10-25, particularly preferably 22,
b=5-30, preferably 10-25, particularly preferably 24, and
c=3.

Further more preferred compositions according to the invention include an organosiloxane-oxyalkylene copolymer of general structural formula (II) having an HLB value in the range of from 8-20, preferably 10-18, particularly preferably 11-16, and with
R¹=methyl,
R²═—C_{c}H_{2c}—O—(C₂H₄O—)ₐ(C₃H₆O—)_{b}R⁵,
R⁵=a hydrogen atom,
n=10-500, preferably 20-400, particularly preferably 50-300,
p=10-50, preferably 15-40, particularly preferably 20-30,
a=5-30, preferably 10-25, particularly preferably 22,
b=5-30, preferably 10-25, particularly preferably 24, and
c=3.

Further more preferred compositions according to the invention include an organosiloxane-oxyalkylene copolymer of general structural formula (II) having an HLB value in the range of from 8-20, preferably 10-18, particularly preferably 11-16, and with
R¹=methyl,
R²═—C_{c}H_{2c}—O—(C₂H₄O—)ₐ(C₃H₆O—)_{b}R⁵,
R⁵=a hydrogen atom,
n=10-500, preferably 20-400, particularly preferably 50-300,
p=10-50, preferably 15-40, particularly preferably 20-30,
a=5-30, preferably 10-25, particularly preferably 22, and
b=5-30
c=3.

Further more preferred compositions according to the invention include an organosiloxane-oxyalkylene copolymer of general structural formula (II) having an HLB value in the range of from 8-20, preferably 10-18, particularly preferably 11-16, and with
R¹=methyl, and
R²═—C_{c}H_{2c}—O—(C₂H₄O—)ₐ(C₃H₆O—)_{b}R⁵
   with
   a=18
   b=18
   c=3
   R⁵=methyl,
   n=10-500, and
   p=10-50.
      Such an organosiloxane-oxyalkylene copolymer is available, for example, under the trade name Dow Corning 190 (INCI: PEG/PPG-18/18 dimethicone).

Further more preferred compositions according to the invention include an organosiloxane-oxyalkylene copolymer of general structural formula (II) having an HLB value in the range of from 8-20, preferably 10-18, particularly preferably 11-16, and with
R¹=methyl,
R²═—C_{c}H_{2c}—O—(C₂H₄O—)ₐ(C₃H₆O—)_{b}R⁵
   with
   a=12,
   b=0,
   c=3, and
   R⁵=methyl,
n=10-500, and
p=10-50.

Such an organosiloxane-oxyalkylene copolymer is for example available under the trade name Dow Corning 193 (INCI: PEG-12 dimethicone). Under certain conditions, Dow Corning 193 (PEG-12 dimethicone) may be olfactorily unstable.

Further more preferred compositions according to the invention include an organosiloxane-oxyalkylene copolymer of general structural formula (II) having an HLB value in the range of from 8-20, preferably 10-18, particularly preferably 11-16, and with
R¹=methyl,
R²═—C_{c}H_{2c}—O—(C₂H₄O—)ₐ(C₃H₆O—)_{b}R⁵
   with
   a=7,
   b=0,
   c=2, and
   R⁵=methyl,
n=0, and
p=1.

Such an organosiloxane-oxyalkylene copolymer is for example available under the trade name Silwet L-77.

Further more preferred compositions according to the invention include an organosiloxane-oxyalkylene copolymer of general structural formula (II) having an HLB value in the range of from 8-20, preferably 10-18, particularly preferably 11-16, and with
R¹=methyl,
R²═—C_{c}H_{2c}—O—(C₂H₄O—)ₐ(C₃H₆O—)_{b}R⁵,
   with
   a=22,
   b=24,
   c=3, and
   R⁵=methyl,
n=10-500, and
p=10-50.

Further more preferred compositions according to the invention include an organosiloxane-oxyalkylene copolymer of general structural formula (II) having an HLB value in the range of from 8-20, preferably 10-18, particularly preferably 11-16, and with
R¹=methyl,
R²═—C_{c}H_{2c}—O—(C₂H₄O—)ₐ(C₃H₆O—)_{b}R⁵
   with
   a=17,
   b=18,
   c=3, and
   R⁵=methyl,
n=10-500, and
p=10-50.
   Such an organosiloxane-oxyalkylene copolymer is available, for example, under the trade name Dow Corning Q2-5220 (INCI: PEG/PPG-17/18 dimethicone).

Further more preferred compositions according to the invention include an organosiloxane-oxyalkylene copolymer of general structural formula (II) with an HLB value in the range from 8-20, preferably 10-18, particularly preferably 11-16, and with
R¹=methyl,
R²═—C_{c}H_{2c}—O—(C₂H₄O—)ₐ(C₃H₆O—)_{b}R⁵
   with
   a=20,
   b=6,
   c=3, and
   R⁵=methyl,
n=10-500, and
p=5-50.
   Such an organosiloxane-oxyalkylene copolymer is available, for example, under the trade name Abil B 88184 (INCI: PEG/PPG-20/6 dimethicone).

Further more preferred compositions according to the invention include an organosiloxane-oxyalkylene copolymer of general structural formula (II) with an HLB value in the range from 8-20, preferably 10-18, particularly preferably 11-16, and with-
R¹=methyl,
R²═—C_{c}H_{2c}—O—(C₂H₄O—)ₐ(C₃H₆O—)_{b}R⁵
   with
   a=14,
   b=4,
   c=3, and
   R⁵=methyl,
n=10-500, and
p=5-50.
   Such an organosiloxane-oxyalkylene copolymer is for example available under the trade name Abil B 8851 (INCI: PEG/PPG-14/4 dimethicone).

Further more preferred compositions according to the invention include an organosiloxane-oxyalkylene copolymer of general structural formula (II) having an HLB value in the range of from 8-20, preferably 10-18, particularly preferably 11-16, and with
R¹=tertiary-butyl,
R²═—C_{c}H_{2c}—O—(C₂H₄—O—)ₐ(C₃H₆O—)_{b}R⁵
   with
   a=11,
   b=0,
   c=3, and
   R⁵═H,
n=0, and

p=1.

Further more preferred compositions according to the invention include an organosiloxane-oxyalkylene copolymer of general structural formula (II) having an HLB value in the range of from 8-20, preferably 10-18, particularly preferably 11-16, and with
R¹=isopropyl (—CH(CH₃)₂),
R²═—C_{c}H_{2c}—O—(C₂H₄O—)ₐ(C₃H₆O—)_{b}R⁵
   with
   a=11,
   b=0,
   c=3, and
   R⁵═H,
n=0, and
p=1.

Further more preferred compositions according to the invention include an organosiloxane-oxyalkylene copolymer of general structural formula (II) having an HLB value in the range of from 8-20, preferably 10-18, particularly preferably 11-16, and with
R¹=methyl,
R²═—CH₂—CH(CH₃)—CH₂—O—(C₂H₄O—)ₐ(C₃H₆O—)_{b}R⁵
   with
   a=8,
   b=0, and
   R⁵═H,
n=0, and
p=1.

Further more preferred compositions according to the invention include an organosiloxane-oxyalkylene copolymer of general structural formula (I) having an HLB value in the range of from 8-20, preferably 10-18, particularly preferably 11-16, and with
R¹=methyl,
R²═—C_{c}H_{2c}—O—(C₂H₄O—)ₐ(C₃H₆O—)_{b}R⁵
   with
   a=20,
   b=20,
   c=3, and
   R⁵=methyl,
m=0, and
n=10-500.
   Such an organosiloxane-oxyalkylene copolymer is for example available under the trade name Abil B 8832 (INCI: Bis-PEG/PPG-20/20 dimethicone).

A further preferred organosiloxane-oxyalkylene copolymer is bis-PEG/PPG-16/16 PEG/PPG-16/16 dimethicone.

Organosiloxane-oxyalkylene copolymers of general structural formula (II) specifically mentioned above which cause better release of active substances compared to organosiloxane-oxyalkylene copolymers of general structural formula (I) are more preferred.

Further more preferred organosiloxane-oxyalkylene copolymers d) according to the invention are chosen from linear polysiloxane-polyoxyalkylene block copolymers, in particular from linear polysiloxane-polyoxyethylene-polyoxypropylene block copolymers. A linear polysiloxane-polyoxyethylene-polyoxypropylene block copolymer with the INCI designation PEG/PPG-22/24 dimethicone is most preferred according to the invention. Such a linear polysiloxane-polyoxyethylene-polyoxypropylene block copolymer is for example available under the trade name Mirasil DMCO (INCI: PEG/PPG-22/24 dimethicone) from the Rhodia company.

Further more preferred compositions according to the invention include an organosiloxane-oxyalkylene copolymer d) chosen from PEG/PPG-18/18 dimethicone, PEG-12 dimethicone, PEG/PPG-22/24 dimethicone, PEG/PPG-17/18 dimethicone, PEG/PPG-20/6 dimethicone, PEG/PPG-14/4 dimethicone, bis-PEG/PPG-16/16 PEG/PPG-16/16 dimethicone, and mixtures thereof.

Even more preferably, compositions according to the invention comprise PEG/PPG-22/24 dimethicone and PEG-12 dimethicone. Further more preferred compositions according to the invention comprise PEG/PPG-22/24 dimethicone and PEG/PPG-20/6 dimethicone. Further more preferred compositions according to the invention comprise PEG/PPG-22/24 dimethicone and PEG/PPG-14/4 dimethicone. Further more preferred compositions according to the invention comprise PEG/PPG-22/24 dimethicone and PEG/PPG-17/18 dimethicone. Further more preferred compositions according to the invention comprise PEG/PPG-22/24 dimethicone and bis-PEG/PPG-20/20 dimethicone. Most preferred are compositions according to the invention comprising PEG/PPG-22/24 dimethicone and PEG/PPG-20/6 dimethicone. Furthermore most preferred compositions according to the invention comprise PEG/PPG-22/24 dimethicone and PEG/PPG-14/4 dimethicone. Furthermore most preferred compositions according to the invention comprise PEG/PPG-22/24 dimethicone and PEG/PPG-17/18 dimethicone.

Further more preferred compositions according to the invention comprise PEG-12 dimethicone and PEG/PPG-20/6 dimethicone. Further more preferred compositions according to the invention comprise PEG-12 dimethicone and PEG/PPG-14/4 dimethicone. Further more preferred compositions according to the invention comprise PEG-12 dimethicone and PEG/PPG-17/18 dimethicone. Further more preferred compositions according to the invention comprise PEG-12 dimethicone and bis-PEG/PPG-20/20 dimethicone. Most preferred are compositions according to the invention that comprise PEG-12 dimethicone and PEG/PPG-20/6 dimethicone. Furthermore, most preferred are compositions according to the invention that comprise PEG-12 dimethicone and PEG/PPG-17/18 dimethicone.

Further more preferred compositions according to the invention comprise PEG/PPG-20/6 dimethicone and PEG/PPG-14/4 dimethicone. Further more preferred compositions according to the invention comprise PEG/PPG-20/6 dimethicone and PEG/PPG-17/18 dimethicone. Further more preferred compositions according to the invention comprise PEG/PPG-20/6 dimethicone and bis-PEG/PPG-20/20 dimethicone.

Further more preferred compositions according to the invention are characterized in that they comprise PEG/PPG-14/4 dimethicone and PEG/PPG-17/18 dimethicone. Further more preferred compositions according to the invention comprise PEG/PPG-14/4 dimethicone and bis-PEG/PPG-20/20 dimethicone. Further more preferred compositions according to the invention comprise PEG/PPG-17/18 dimethicone and bis-PEG/PPG-20/20 dimethicone.

Further preferred compositions according to the invention comprise an organosiloxane-oxyalkylene copolymer d) having a water solubility of at least 2 g per 100 g of aqueous solution. Further preferred compositions according to the invention comprise an organosiloxane-oxyalkylene copolymer d) having a water solubility of at least 5 g per 100 g of aqueous solution. Further preferred compositions according to the invention comprise an organosiloxane-oxyalkylene copolymer d) miscible with water to at least 2% by weight. Further preferred compositions according to the invention comprise an organosiloxane-oxyalkylene copolymer d) miscible with water to at least 5% by weight. All details concerning water solubility and water miscibility given above refers to a temperature of 20 ° C. and a pressure of 1013.25 mbar.

Further preferred compositions according to the invention have at least one organosiloxane-oxyalkylene copolymer present in an amount of 0.01-5% by weight, preferably 0.1-4% by weight, particularly preferably in a total amount of 0.5-3% by weight, most preferably in a total amount of 1-2% by weight, based on total weight of the composition.

In a preferred embodiment according to the invention, the anti-transpirant active substances and optionally further insoluble active substances in the carrier are suspended in at least one oil which is liquid under normal conditions. For better applicability, at least one lipophilic thickener is further added as suspension adjuvant to this suspension. The addition of such a lipophilic thickener is in particular preferred for liquid and/or suspension-like administration forms, in particular for roll-on compositions. Further preferred compositions according to the invention are therefore characterized in that they contain at least one lipophilic thickener. Preferred compositions according to the invention are characterized in that said at least one lipophilic thickener is selected from hydrophobicized clay minerals, pyrogenic silicic acids, bentone gels, ethylene/propylene/styrene copolymers, butylenes/ethylene/styrene copolymers, dextrin esters, silicone elastomers, waxes solid under normal conditions and/or glycerol triesters. Among them, hydrophobicized clay minerals are more preferred. The compositions according to the invention contain in a preferred embodiment at least one suspension or thickening agent. Particularly suitable thickeners are hydrophobicized clay minerals such as montmorillonites, hectorites and bentonites, in particular disteardimonium hectorite and quaternium-18 hectorite. The commercial thickeners provide these hydrophobicized clay minerals in the form of a gel in cyclomethicone and if desired in the form of a gel activator, such as for example propylene-carbonate. Further suitable thickeners are pyrogenic silicic acids, for example, the commercial products of the Aerosil® series of Degussa.

Preferred hydrophobicized clay minerals include hydrophobicized montmorillonites, hydrophobicized hectorites and hydrophobicized bentonites, particularly preferably from disteardimonium hectorite, stearalkonium hectorite, quaternium-18 hectorite and quaternitun-18 bentonite. Commercial thickeners provide these hydrophobicized clay minerals in the form of a gel in an oil component, preferably in cyclomethicone and/or a non-silicone oil component, such as propylene-carbonate. Such gels are available, for example, under the trade name Bentone® or Thixogel. Preferred compositions according to the invention contain at least one hydrophobicized clay mineral in a total amount of 0.5-10% by weight, preferably 1-7% by weight, particularly preferably 2-6% by weight, most preferably 3-5% by weight, based on total weight of the composition. Such hydrophobicized clay minerals usually require as an activator, ethanol or propylene-carbonate in an amount of 0.3-3% by weight, preferably 0.5-2% by weight, based on total weight of the composition. Further preferred lipophilic thickeners according to the invention include pyrogenic silicic acids, for example, the commercial products of the Aerosil® series from Evonik Degussa. Hydrophobicized pyrogenic silicic acids, more preferred silica silylates and silica dimethyl silylates, are more preferred. Preferred compositions according to the invention contain at least one pyrogenic silicic acid, preferably at least one hydrophobicized pyrogenic silicic acid, in a total amount of 0.5-10% by weight, preferably 0.8-5% by weight, particularly preferably 1-4% by weight, most preferably 1.5-2% by weight, each based on the total weight of the composition according to the invention. Further preferred compositions according to the invention contain at least one hydrophobicized pyrogenic silicic acid and at least hydrophilic silicic acid.

Further preferred lipophilic thickeners according to the invention are chosen from ethylene/propylene/styrene copolymers and butylene/ethylene/styrene copolymers. The copolymers are particularly preferably applied as a pre-thickened oil-based gel. Preferred compositions according to the invention contain at least one ethylene/propylene/styrene copolymer and/or butylene/ethylene/styrene copolymer in an amount of 0.05-3% by weight, preferably 0.1-2% by weight, particularly preferably 0.2-1.0% by weight, most preferably 0.3-0.5% by weight, based on total weight of the composition.

Further preferred lipophilic thickeners according to the invention are selected from silicone elastomers. A further preferred embodiment of the invention is characterized in that at least one silicone elastomer, which may be obtained by cross-linking of an organopolysiloxane, which contains at least 2 C₂-C₁₀ alkenyl groups with a terminal double bond in each molecule, with an organopolysiloxane, which has at least 2 silicone-bound hydrogen atoms in each molecule, is contained.

More preferred organopolysiloxanes according to the invention with at least 2 C₂-C₁₀ alkenyl groups with a terminal double bond in the molecule are selected from methylvinylsiloxanes, methylvinylsiloxane-dimethylsiloxane copolymers, dimethylpolysiloxanes with dimethylvinyl-siloxy end groups, dimethylsiloxane-methylphenylsiloxane copolymers with dimethylvinylsiloxy end groups, dimethylsiloxane-diphenylsiloxane-methylvinylsiloxane copolymers with dimethylvinylsiloxy end groups, dimethylsiloxane-methylvinylsiloxane copolymers with trimethylsiloxy end groups, dimethylsiloxane-methylphenylsiloxane-methylvinylsiloxane copolymers with trimethylsiloxy end groups, methyl-(3,3,3-trifluoropropy1)-polysiloxanes with dimethylvinylsiloxy end groups, and dimethylsiloxane-methyl-(3,3,3-trifluoropropy1)-siloxane copolymers with dimethylvinylsiloxy end groups.

More preferred cross-linking organopolysiloxanes according to the invention with at least two silicone-bound hydrogen atoms are selected from methylhydrogen-polysiloxanes with trimethylsiloxy end groups, dimethylsiloxane-methylhydrogensiloxane copolymers with trimethylsiloxy end groups and cyclic dimethylsiloxane-methylhydrogen-siloxane copolymers.

More preferred silicone elastomers according to the invention, which exist in a swollen state as a raw material already in silicone liquid at room temperature under normal conditions and represent a silicone-based gel, are commercially available, for example under the trade name SFE 168, a cyclomethicone (and) dimethicone/vinyl dimethicone cross-polymer from GE Silicones, vinyl dimethicone cross-polymers, contained in KSG-15 (cyclomethicone (and) dime-thicone/vinyl dimethicone cross-polymer, silicone elastomer content 4-10% by weight), KSG-16 (dimethicone (and) dimethicone/vinyl dimethicone cross-polymer, silicone elastomer content 20-30% by weight), KSG-17 (cyclomethicone (and) dimethicone/vinyl dimethicone cross-polymer), KSG-18 (phenyl trimethicone (and) dimethicone/phenyl vinyl dimethicone cross-polymer, silicone elastomer content 10-20% by weight); and KSG-20, available from Shin Etsu Silicones of America (Akron, Ohio), and from Grant Industries Inc. (Elmwood Park, N.J.) the products from the Gransil®series.

A further preferred embodiment of the invention is characterized in that the silicone elastomer may be obtained by cross-linking of an organopolysiloxane, which contains at least 2 C₂-C₁₀-alkenyl groups, with a terminal double bond in each molecule, with at least one alpha, omega-diene. More preferred cross-linking alpha, omega-dienes according to the invention have the formula CH₂═CH(CH₂)ₓCH═CH₂ with x=1-20. More preferred alpha, omega-dienes are selected from 1,4-pentadiene, 1,5-hexadiene, 1,6-heptadiene, 1,7-octadiene, 1,8-nonadiene, 1,11-dodecadiene, 1,13-tetradecadiene and 1,19-eicosadiene.

Preferred compositions according to the invention contain at least one silicone elastomer in a total amount of 0.05-3% by weight, preferably 0.1-2% by weight, particularly preferably 0.15-0.5% by weight, most preferably 0.2-0.3% by weight each based on the total weight of the composition according to the invention.

Further preferred compositions according to the invention contain at least one silicone gum. Surprisingly, it was noticed that by adding a silicone gum, the deodorant effect of the compositions according to the invention may be further prolonged. Without the intention of being bound to this theory, it is assumed that the silicone gum forms a film on the skin, through which the antiperspirant active substance particles better adhere to the skin.

A more preferred silicone gum according to the invention is silicone polymers with the INCI designation: dimethiconol. These dimethiconols are preferably applied in a lowly-concentrated solution in cyclomethicone or dimethicone with a kinematic viscosity from 0.65 cSt up to a maximum of 10 cSt. More preferred dimethiconols are available from Dow Corning under the trade names Dow Corning 1401, Dow Coming 1403 and Dow Corning 1501; these products contain 10 to 13% by weight of dimethiconol in cyclomethicone or dimethicone.

Preferred compositions according to the invention contain at least one silicone gum in a total amount of 0.01-0.5% by weight, preferably 0.05-0.2% by weight, particularly preferably 0.1-0.15% by weight, based on total weight of the composition.

Compositions according to the invention contain at least one antiperspirant active substance. Preferred antiperspirant active substances are selected from astringent water-soluble inorganic and organic salts of aluminum, zirconium and zinc or any mixtures of these salts. According to the invention, water-solubility refers to a solubility of at least 5% by weight at 20° C. (i.e., at least 5 g of antiperspirant active substance is soluble in 95 g of water at 20° C.).

More preferred antiperspirant active substances include aluminum hydroxychloride, in particular aluminum hydroxychloride with general formula [Al₂(OH)₅Cl.1-6 H₂O]ₙ, preferably [Al₂(OH)₅Cl.2-3 H₂O]ₙ, which may exist in a non-activated or in an activated (depolymerized) form, as well as aluminum hydroxychloride with the general formula [Al₂(OH)₄Cl₂.1-6 H₂O]ₙ, preferably [Al₂(OH)₄Cl₂.2-3 H₂O]ₙ, which may exist in a non-activated or in an activated (depolymerized) form. The preparation of preferred antiperspirant active substances is disclosed for example in U.S. Pat. No. 3,887,692, U.S. Pat. No. 3,904,741, U.S. Pat. No. 4,359,456, GB 2048229 and GB 1347950.

Further the following compounds are preferred: aluminum sesquichlorohydrate, aluminum dichlorohydrate, aluminum chlorohydrex-propyleneglycol (PG) or aluminum chlorohydrex-poly-ethyleneglycol (PEG), aluminum- or aluminum-zirconium-glycol complexes, for example aluminum- or aluminum-zirconium-propyleneglycol complexes, aluminumsesquichlorohydrex-PG or aluminumsesquichlorohydrex-PEG, aluminum-PG-dichlorohydrex or aluminum-PEG-dichlorohydrex, aluminum hydroxide, further selected from aluminum-zirconium chlorohydrates, such as aluminum-zirconium trichlorohydrate, aluminum-zirconium tetrachlorohydrate, aluminum-zirconium pentachlorohydrate, aluminum-zirconium octachlorohydrate, aluminum-zirconium-chlorohydrate-glycine complexes such as aluminum-zirconiumtrichlorohydrexglycine, aluminum-zirconium tetrachlorohydrexglycine, aluminum-zirconium pentachlorohydrexglycine, aluminumzirconium octachlorohydrexglycine, potassiumaluminum sulfate (ICARSO₄)₂.12 H₂O, alum), aluminum undecylenoyl-collagenamino acid, sodium aluminum lactate+aluminum-sulfate, sodiumaluminum chlorohydroxylactate, aluminum bromohydrate, aluminum chloride, complexes of zinc and sodium salts, complexes of lanthanum and cerium, aluminum salts of lipoamino acids, aluminum sulfate, aluminum lactate, aluminum chlorohydroxyallantoinate, sodium-aluminum chlorohydroxylactate, zinc chloride, zinc sulfocarbolate, zinc sulfate, zirconyl oxyhalides, in particular zirconyl oxychlorides, zirconyl hydroxyhalides, in particular zirconyl hydroxychlorides (zirconium chlorohydrate).

More preferred antiperspirant active substances according to the invention are selected from so-called “activated” aluminum and aluminum-zirconium salts, which are also designated as antiperspirant active substances “with increased activity (enhanced activity)”. Such active substances are known from the state of the art and are also commercially available. Their preparation is disclosed, for example, in GB 2048229, U.S. Pat. No. 4,775,528 and U.S. Pat. No. 6,010,688. Activated aluminum and aluminum-zirconium salts are generally produced by heat treatment of a relatively diluted solution of the salt (for example about 10% by weight of salt), in order to increase its HPLC-Peak 4-to-Peak 3-area ratio. The activated salt may subsequently be dried into a powder, in particular be spray-dried. In addition to spray-drying, also roller drying is suitable for example. Activated aluminum and aluminum-zirconium salts typically have a HPLC-Peak 4-to-Peak 3-area ratio of at least 0.4, preferably at least 0.7, particularly preferably at least 0.9, wherein at least 70% of the aluminum are associated with these peaks.

Activated aluminum and aluminum-zirconium salts are not necessarily to be applied as a spray-dried powder. Equally preferred antiperspirant active substances according to the invention are non-aqueous solutions or solubilisates of an activated antiperspirant aluminum or aluminum-zirconium salt, for example according to U.S. Pat. No. 6,010,688, which, by adding an effective amount of a polyhydric alcohol, which has 3-6 carbon atoms and 3-6 hydroxyl groups, preferably propyleneglycol, sorbitol and pentaerythritol, are stabilized against the loss of activation against the fast degradation of the HPLC-Peak 4:Peak 3-area ratio of the salt. Compositions are for example preferred, which contain in weight percent (USP): 18-45% by weight of an activated aluminum or aluminum-zirconium salt, 55-82% by weight of at least one anhydrous polyhydric alcohol with 3-6 carbon atoms and 3-6 hydroxyl groups, preferably propyleneglycol, butyleneglycol, diethyleneglycol, dipropyleneglycol, glycerol, sorbitol and pentaerythritol, particularly preferably propyleneglycol.

More preferred are also complexes of activated antiperspirant aluminum or aluminum-zirconium salt with a polyhydric alcohol, which contain 20-50% by weight, particularly preferably 20-42% by weight, of activated antiperspirant aluminum or aluminum-zirconium salt and 2-16% by weight of molecular bound water, wherein the balance to 100% by weight is at least one polyhydric alcohol with 3-6 carbon atoms and 3-6 hydroxyl groups. Propyleneglycol, propyleneglycol/sorbitol mixtures and propyleneglycol/pentaerythritol mixtures are preferred alcohols of this type. Such preferred complexes according to the invention of an activated antiperspirant aluminum or aluminum-zirconium salt with a polyhydric alcohol are for example disclosed in U.S. Pat. No. 5,643,558 and U.S. Pat. No. 6,245,325.

Further preferred antiperspirant active substances are basic calcium-aluminum salts as they are disclosed for example in U.S. Pat. No. 2,571,030. These salts are prepared by reacting calcium carbonate with aluminum chlorohydroxide or aluminum chloride and aluminum powder or by adding calcium chloride dihydrate to aluminum chlorohydroxide.

Further preferred antiperspirant active substances are aluminum-zirconium complexes, as disclosed, for example, in U.S. Pat. No. 4,017,599, which are buffered with salts of aminoacids, in particular with alkali and earth alkali glycinates.

Further preferred antiperspirant active substances are activated aluminum or aluminum-zirconium salts, as disclosed, for example, in U.S. Pat. No. 6,245,325 or U.S. Pat. No. 6,042,816, containing 5-78% by weight (USP) of an activated antiperspirant aluminum or aluminum-zirconium salt, an aminoacid or a hydroxyalkanoic acid in such an amount so as to provide a (amino acid or hydroxyalkanoic acid)-to-(Al+Zr) weight ratio of 2:1-1:20 and preferably 1:1 to 1:10, as well as a water-soluble calcium salt in such an amount so as to provide a Ca:(Al+Zr) weight ratio of 1:1-1:28 and preferably 1:2-1:25.

Most preferred solid activated antiperspirant salt compositions, for example, according to U.S. Pat. No. 6,245,325 or U.S. Pat. No. 6,042,816, contain 48-78% by weight (USP), preferably 66-75% by weight of an activated aluminum or aluminum-zirconium salt and 1-16% by weight, preferably 4-13% by weight of molecular bound water (hydration water), furthermore as much waster-soluble calcium salt so that the Ca:(Al+Zr) weight ratio is 1:1-1:28, preferably 1:2-1:25, and as much aminoacid so that the aminoacid-to-(Al+Zr) weight ratio is 2:1-1:20, preferably 1:1-1:10.

Further more preferred solid antiperspirant activated salt compositions, for example, according to U.S. Pat. No. 6,245,325 or U.S. Pat. No. 6,042,816, contain 48-78% by weight (USP), preferably 66-75% by weight of an activated aluminum or aluminum-zirconium salt and 1-16% by weight, preferably 4-13% by weight of molecular bound water (hydration water) further as much water-soluble calcium salt, so that the Ca:(Al+Zr) weight ratio is 1:1-1:28, preferably 1:2-1:25, and as much glycine, so that the glycine-to-(Al+Zr) weight ratio is 2:1-1:20, preferably 1:1-1:10.

Further more preferred solid antiperspirant activated salt compositions, for example according to U.S. Pat. No. 6,245,325 or U.S. Pat. No. 6,042,816, contain 48-78% by weight (USP), preferably 66-75% by weight of an activated aluminum or aluminum-zirconium salt, and 1-16% by weight, preferably 4-13% by weight of molecular bound water, further as much water-soluble calcium salt, so that the Ca:(Al+Zr) weight ratio is 1:1-1:28, preferably 1:2-1:25, and as much hydroxyalkanoic acid, so that the hydroxyalkanoic acid-to-(Al+Zr) weight ratio is 2:1-1:20, preferably 1:1-1:10.

Water-soluble calcium salts preferred for stabilizing the antiperspirant salts include calcium chloride, calcium bromide, calcium nitrate, calcium citrate, calcium formate, calcium acetate, calcium gluconate, calcium ascorbate, calcium lactate, calcium glycinate, calcium carbonate, calcium sulfate, calcium hydroxide, as well as mixtures thereof.

Preferred aminoacids for stabilizing antiperspirant salts include glycine, alanine, leucine, isoleucine, β-alanine, valine, cysteine, serine, tryptophane, phenylalanine, methionine, β-amino-n-butanoic acid and γ-amino-n-butanoic acid and salts thereof, respectively in the d-form, the i-form and the dl-form; glycine is particularly preferred.

Preferred hydroxyalkanoic acids for stabilizing antiperspirant salts include glycolic acid and lactic acid.

Further preferred antiperspirant active substances are activated aluminum or aluminum-zirconium salts, as they are disclosed for example in U.S. Pat. No. 6,902,723, containing 5-78% by weight (USP) of an activated antiperspirant aluminum or aluminum-zirconium salt, an aminoacid or hydroxyalkanoic acid in such an amount in order to provide a (aminoacid or hydroxyalkanoic acid)-to-(Al+Zr) weight ratio of 2:1-1:20 and preferably 1:1 to 1:10, as well as a water-soluble strontium salt in such an amount in order to provide a Sr:(Al+Zr) weight ratio of 1:1-1:28 and preferably 1:2-1:25.

More preferred solid antiperspirant activated salt compositions, for example, according to U.S. Pat. No. 6,902,723, contain 48-78% by weight (USP), preferably 66-75% by weight of an activated aluminum or aluminum-zirconium salt and 1-16% by weight, preferably 4-13% by weight of molecular bound water, further as much water-soluble strontium salt so that the Sr:(Al+Zr) weight ratio is 1:1-1:28, preferably 1:2-1:25, and as much aminoacid so that the amino acid-to-(Al+Zr) weight ratio is 2:1-1:20, preferably 1:1-1:10.

Further more preferred solid antiperspirant activated salt compositions, for example, according to U.S. Pat. No. 6,902,723, contain 48-78% by weight (USP), preferably 66-75% by weight of an activated aluminum or aluminum-zirconium salt and 1-16% by weight, preferably 4-13% by weight of molecular bound water, further as much water-soluble strontium salt so that the Sr:(Al+Zr) weight ratio is 1:1-1:28, preferably 1:2-1:25, and as much glycine so that the glycine-to-(Al+Zr) weight ratio is 2:1-1:20, preferably 1:1-1:10.

Further more preferred solid antiperspirant activated salt compositions, for example, according to U.S. Pat. No. 6,902,723, contain 48-78% by weight (USP), preferably 66-75% by weight of an activated aluminum or aluminum-zirconium salt and 1-16% by weight, preferably 4-13% by weight of molecular bound water, further as much water-soluble strontium salt, so that the Sr:(Al+Zr) weight ratio is 1:1-1:28, preferably 1:2-1:25, and as much hydroalkanoic acid, so that the hydroxyalkanoic acid-to-(Al+Zr) weight ratio is 2:1-1:20, preferably 1:1-1:10.

Further activated aluminum salts are those of general formula Al₂(OH)₆₋ₐXa, wherein X is Cl, Br, I or NO₃ and “a” is a value from 0.3 to 5, preferably from 0.8 to 2.5 and particularly preferably 1 to 2, so that the molar ratio of Al:X is 0.9:1 to 2.1:1, as they are disclosed for example in U.S. Pat. No. 6,074,632. In these salts, some hydration water is associatively bound generally, typically 1 to 6 moles of water per mole of salt. Aluminum chlorohydrate is more preferred (i.e., X is Cl in the aforementioned formula) as well as special 5/6 basic aluminum chlorohydrate, wherein “a” is 1, so that the molar ratio of aluminum to chlorine is 1.9:1 to 2.1:1.

Preferred activated aluminum-zirconium salts include mixtures or complexes of the aluminum salts described above with zirconium salts of formula ZrO(OH)_{2-pb}Y_{b}, wherein Y is Cl, Br, I, NO₃ or SO₄, b is a rational number from 0.8 to 2 and p is the valence of Y, as they are disclosed for example in U.S. Pat. No. 6,074,632. The zirconium salts generally also have some hydration water associatively bound, typically 1 to 7 moles of water per mole of salt. Preferably, the zirconium salt is zirconyl hydroxychloride with formula ZrO(OH)_{2-b}Cl_{b}, wherein b is a rational number from 0.8 to 2, preferably 1.0 to 1.9. Preferred aluminum-zirconium salts have a molar Al:Zr ratio from 2 to 10 and a metal:(X+Y) ratio from 0.73 to 2.1, preferably 0.9 to 1.5. A more preferred salt is aluminum-zirconium chlorohydrate (i.e., X and Y are CI), which has a molar Al:Zr ratio from 2 to 10 and a molar metal:Cl ratio from 0.9 to 2.1. The term aluminum-zirconium chlorohydrate encompasses the tri-, tetra-, penta- and octa-chlorohydrate forms.

Preferred zirconium salts according to the invention have the general formula ZrO(OH)₂₋ₐClₐ.x H₂O with a=1.5-1.87; x=1-7, wherein a and x are rational numbers. These zirconium salts are disclosed, for example, in Belgian document BE 825146.

Further preferred antiperspirant active substances are disclosed in U.S. Pat. No. 6,663,854 and US 20040009133.

Antiperspirant active substances may exist both in solubilized form and also in an undissolved suspended form.

Insofar that the antiperspirant active substances exist suspended in a carrier which is not miscible with water, for reasons of product stability it is preferred that the active substance particles have a number average particle size of 0.1-200 μm, preferably 1-50 μm, particularly preferably 3-20 μm and most preferably 5-10 μm. Preferred active substance particles have a volume average particle size of 0.2-220 μm, preferably 3-60 μm, more preferred 4-25 μm and most preferred 10-15.5 μm.

Preferred aluminum salts and aluminum-zirconium salts have a molar metal-to-chloride ratio of 0.9-2.0, preferably 1.0-1.51, particularly preferably 1.1-1.5, most preferably 1.3-1.4.

Preferred aluminum-zirconium chlorohydrates have the empirical formula AlₙZr(OH)_{[3n+4-m(n+1)]}(Cl)_{[m(n+1)]} with n=2.0-10.0, preferably 3.0-8.0, m=0.77-1.11 (corresponding to a molar metal (Al+Zr)-to-chloride ratio of 1.3-0.9), preferably m=0.91-1.11 (corresponding to M:Cl=1.1-0.9), and particularly preferably m=1.00-1.11 (corresponding to M:Cl=1.0-0.9), further very preferably m=1.02-1.11 (corresponding to M:Cl=0.98-0.9) as well as very preferably m=1.04-1.11 (corresponding to M:Cl=0.96-0.9).
Further preferred aluminum-zirconium trichlorohydrates have the empirical formula Al₄(OH)₁₀Cl₂.Zr(OH)Cl.
Further preferred aluminum-zirconium tetrachlorohydrates have the empirical formula Al₄(OH)₁₀Cl₂.ZrCl₂.
Further preferred aluminum-zirconium pentachlorohydrates have the empirical formula Al₈(OH)₂₀Cl₆.Zr(OH)Cl.
Further preferred aluminum-zirconium octachlorohydrates have the empirical formula Al₈(OH)₁₈Cl₆.Zr(OH)Cl.

Generally some hydration water is associatively bound in these salts, typically 1-6 moles of water per mole of salt, corresponding to 1-30% by weight, preferably 4-13% by weight of hydration water.

Preferred antiperspirant salts are aluminum-zirconium tetrachlorohydrates (molar ratio Al:Zr=2-6; M:Cl=0.9-1.3, preferably 0.95:1.3, more preferably 1.0:1.1), in particular salts with a molar metal-to-chloride ratio of 0.9-1.1, preferably 0.9-1.0.

Further preferred antiperspirant salts are aluminum chlorohydrates with a molar metal-to-chloride ratio of M:Cl=1.9-2.1.

Preferred aluminum-zirconium chlorohydrates are usually associated with an aminoacid, in order to prevent polymerization of the zirconium species during the preparation. Preferred stabilizing aminoacids include glycine, alanine, leucine, isoleucine, β-alanine, cysteine, valine, serine, tryptophane, phenylalanine, methionine, β-amino-n-butanoic acid, and ₁-amino-n-butanoic acid and salts thereof, respectively in the d-form, the 1-form and the dl-form; glycine is more preferred. The aminoacid is contained in an amount of 1-3 moles, preferably 1.3-1.8 moles, each time per mole of zirconium in the salt.

The aforementioned aluminum-zirconium trichlorohydrates, aluminum-zirconium tetrachlorohydrates, aluminum-zirconium pentachlorohydrates and aluminum-zirconium octachlorohydrates exist both activated and also non-activated, preferably as a complex with glycine. More preferred antiperspirant active substances include activated aluminum-zirconium-trichlorohydrex glycine, in particular activated aluminum-zirconium trichlorohydrex glycine with an active substance free of crystal water and free of glycine (USP) of 69.5-88% by weight, preferably 72-85% by weight, particularly preferably 77-80% by weight, based on raw material as such with a molar metal:Cl ratio from 0.9 to 1.5 and a molar Al:Zr ratio of 3.4-3.8.

Further more preferred antiperspirant active substances include non-activated aluminum-zirconium trichlorohydrex glycine, in particular non-activated aluminum-zirconium-trichlorohydrex glycine, with an active substance free of crystal water and free of glycine (USP) of 69.5-88% by weight, preferably 72-85% by weight, more preferred 77-80% by weight, based on raw material as such, with a molar metal:Cl ratio from 0.9 to 1.5 and a molar Al:Zr ratio of 3.4-3.8.

Further more preferred antiperspirant active substances include activated aluminum-zirconium tetrachlorohydrex glycine, in particular activated aluminum-zirconium tetrachlorohydrex glycine with an active substance free of crystal water and free of glycine (USP) of 72-88% by weight, preferably 77-85% by weight, based on raw material as such, with a molar metal:Cl ratio from 0.9 to 1.5 and a molar Al:Zr ratio of 3.4-3.8.

Further more preferred antiperspirant active substances include non-activated aluminum-zirconium tetrachlorohydrex glycine, in particular non-activated aluminum-zirconium-tetrachlorohydrex glycine with an active substance free from crystal water and free from glycine (USP) of 72-88% by weight, preferably 77-85% by weight, each based on the raw material as such, a molar metal:Cl ratio from 0.9 to 1.5 and a molar Al:Zr ratio of 3.4-3.8.

Further more preferred antiperspirant active substances include activated aluminum-zirconium pentachlorohydrex glycine, in particular activated aluminum-zirconiumpentachlorohydrex glycine with an active substance (USP) free of crystal water and free of glycine of 72-88% by weight, preferably 77-86% by weight, particularly preferably 78-81.5% by weight, based on raw material as such, a molar metal:Cl ratio from 1.51 to 2.0 and a molar Al:Zr ratio of 9.2-9.8. Further more preferred antiperspirant active substances include non-activated aluminum-zirconium pentachlorohydrex glycine, in particular non-activated aluminum-zirconium pentachlorohydrex glycine with an active substance (USP) free of crystal water and free of glycine, of 72-88% by weight, preferably 77-86% by weight, particularly preferably 78-81.5% by weight, based on raw material as such, a molar metal:Cl ratio from 1.51 to 2.0 and a molar Al:Zr ratio of 9.2-9.8.

Crystal water content for the aforementioned activated as well as non-activated aluminum-zirconium trichlorohydrex glycines, aluminum-zirconium tetrachlorohydrex glycines, aluminum-zirconium pentachlorohydrex glycines and aluminum-zirconium octachlorohydrex glycines is 1.5-20% by weight, preferably 7-15% by weight, based on the raw material as such.

Further preferred aluminum-zirconium trichlorohydrex glycines have the empirical formula [Al₄(OH)₁₀Cl₂.Zr(OH)Cl].NH₂CH₂COOH.
Further preferred aluminum-zirconium tetrachlorohydrex glycines have the empirical formula [Al₄(OH)₁₀Cl₂.ZrOCl₂].NH₂CH₂COOH.
Further preferred aluminum-zirconium pentachlorohydrex glycines have the empirical formula [Al₈(OH)₂₀Cl₄.Zr(OH)Cl].NH₂CH₂COOH.
Further preferred aluminum-zirconium octachlorohydrex glycines have the empirical formula [Al₈(OH)₁₈Cl₆.Zr(OH)Cl].NH₂CH₂COOH or [Al₈(OH)₁₈Cl₆.ZrOCl₂].NH₂CH₂COOH.

Aluminum-zirconium chlorohydrate-glycine salts are further preferred according to the invention, which are stabilized with betaine ((CH₃)₃N⁺—CH₂—COO⁻). More preferred corresponding compounds have a total molar (betaine+glycine)/Zr ratio of (0.1-3.0):1, preferably (0.7-1.5):1 and a molar ratio of betaine to glycine of at least 0.001:1. Corresponding compounds are, for example, disclosed in U.S. Pat. No. 7,105,691.

A so-called “activated” salt is contained in a more preferred embodiment according to the invention, as a particularly effective antiperspirant salt, in particular one with a high HPLC-peak 5 aluminum content, in particular with a peak 5 area of at least 33%, particularly preferably at least 45%, based on the total surface area under the peaks 2-5, as measured with HPLC, of a 10% by weight aqueous solution of the active substance under conditions, under which the aluminum species are separated in at least four successive peaks (designated by peaks 2-5). Preferred aluminum-zirconium salts with a high HPLC-peak 5 aluminum content (also designated as “E⁵AZCH”) are for example disclosed in U.S. Pat. No. 6,436,381 and U.S. Pat. No. 6,649,152. Further such activated “E⁵AZCH” salts are preferred, for which the HPLC-peak 4-to-peak 3 surface area ratio is at least 0.4, preferably at least 0.7, particularly preferably at least 0.9.

Further more preferred antiperspirant active substances are such aluminum-zirconium salts with a high HPLC-peak 5 aluminum content, which are additionally stabilized with a water-soluble strontium salt and/or with a water-soluble calcium salt. Corresponding salts are, for example, disclosed in U.S. Pat. No. 6,923,952.

Further antiperspirant active substances are selected from astringent titanium salts, as, disclosed, for example, in GB 2299506 A.

The antiperspirant active substances may be applied as non-aqueous solutions or as glycolic solubilisates.

More preferred compositions according to the invention comprise said at least one antiperspirant active substance in an amount of 5-40% by weight, preferably 10-35% by weight, particularly preferably 11-28% by weight and most preferably 12-20% by weight, based on total weight of the active substance free of crystal water and free of ligands (USP) in the total composition.

In a more preferred embodiment, the composition contains an astringent aluminum salt, in particular aluminum chlorohydrate, particularly preferably aluminum chlorohydrate with an active substance (USP) free of crystal water of 72-88% by weight, based on raw material as such. Preferred non-activated aluminum chlorohydrates include those marketed in powder form such as Micro Dry®, Micro Dry® Ultrafine or Micro Dry®-323 from Summit Reheis, as Chlorhydrol® (powder), as well as in an activated form as Reach® 101, Reach® 103, Reach® 501 from Reheis/Summit or AACH-7171 from Summit. An aluminum-sesquichlorohydrate from Reheis is commercialized under the designation of Reach® 301, which is also more preferred.

Aluminum-zirconium-tetrachlorohydrex-glycine complexes are more preferred, which are commercially available as powders, for example from Summit Reheis under the designation Rezal® 36 GP, Summit AZG-369 or Summit AZG-364, or, in an activated quality, as Summit Reach® AZP-908.

Further aluminum-zirconium-pentachlorohydrex-glycine complexes are more preferred, which for example are commercially available in an activated quality from Summit under the designations AAZG-3108 and AAZG-3110 as powders.

Compositions according to the invention contain at least one oil as a carrier fluid. Preferred antiperspirant compositions according to the invention contain 30-95% by weight, preferably 40-93% by weight, particularly preferably 50-90% by weight, most preferably 55-85% by weight, based on total weight of the composition, of at least one cosmetic oil liquid under normal conditions. Also, a total amount of cosmetic oils liquid under normal conditions of 60, 63, 65, 68, 70, 73, 75, 78 or 80% by weight, based on total weight of the composition, may be more preferred according to the invention, wherein a total amount of 68-73% by weight is more preferred.

In cosmetic oils, a distinction is made between volatile and non-volatile oils. Non-volatile oils refer to oils having a vapor pressure less than 2.66 Pa (0.02 mm Hg) at 20° C. and at an ambient pressure of 1,013 hPa. Volatile oils refer to oils having a vapor pressure of 2.66 Pa-40,000 Pa (0.02 mm-300 mm Hg), preferably 13-12,000 Pa (0.1-90 mm Hg), particularly preferably 15-8,000 Pa, most preferably 300-3,000 Pa, at 20° C. and at an ambient pressure of 1,013 hPa.

Cosmetic oils preferred according to the invention include silicone oils, among which are counted for example dialkyl- and alkylarylsiloxanes, such as cyclopentasiloxane, cyclohexasiloxane, dimethylpolysiloxane, low molecular phenyl trimethicone and methylphenylpolysiloxane, but also hexamethyldisiloxane, octamethyltrisiloxane and decamethyltetrasiloxane. Volatile silicone oils are more preferred, which may be cyclic, such as octamethylcyclotetrasiloxane, decamethylcyclopentasiloxane and dodecamethylcyclohexasiloxane or mixtures thereof, as they are contained for example in the commercial products DC 244, 245, 344 and 345 of Dow Corning (vapor pressure at 20° C. about 13-15 Pa). Volatile linear silicone oils are also more preferred, having 2-10 siloxane units, in particular hexamethyldisiloxane (L₂), octamethyltrisiloxane (L₃), decamethyltetrasiloxane (L₄) as well as any two-compound or three-compound mixtures of L₂, L₃ and/or L₄, preferably mixtures found, for example, in the commercial products Dow Corning® 2-1184, Dow Corning® 200 (0.65 cSt) and Dow Corning® 200 (1.5 cSt) from Dow Corning. A further preferred volatile silicone oil is a low molecular phenyl trimethicone with a vapor pressure at 20° C. of about 2,000 Pa, as this is available for example from GE Bayer Silicones/Momentive under the designation Bayilone Fluid PD 5.

Volatile silicone oils are excellent suitable carrier oils for preferred antiperspirant compositions according to the invention, since they lend them a pleasant skin feeling and minor clothing staining. More preferred antiperspirant compositions according to the invention therefore contain at least one volatile silicone oil in an amount of 30 95% by weight, preferably 40-93% by weight, particularly preferably 50-90% by weight, most preferably 55-85% by weight, based on total weight of the composition.

In addition to or instead of said at least one volatile silicone oil, at least one volatile non-silicone oil may also be contained. Preferred volatile non-silicone oils include C₈-C₁₆ isoparaffins, in particular from isodecane, isoundecane, isododecane, isotridecane, isotetradecane, isopentadecane, and isohexadecane, as well as mixtures thereof. C₁₀-C₁₃ isoparaffin mixtures are preferred, in particular those with a vapor pressure of about 300-400 Pa, preferably 360 Pa at 20° C. Also, the at least one volatile non-silicone oil is preferably contained in a total amount of 30-95% by weight, preferably 40-93% by weight, particularly preferably 50-90% by weight, most preferably 55-85% by weight, based on total weight of the composition.

Because of the drier skin sensation and the faster active substance release, volatile silicone oils, isoparaffins, in particular, isodecane, isoundecane, isododecane, isotridecane, isotetradecane, isopentadecane, isohexadecane and isoeicosane, as well as mixtures of the volatile silicone oils and isoparaffins, in particular isododecane, isohexadecane or isoeicosan, are more preferred as a carrier oil.

Preferred compositions according to the invention are characterized in that said at least one carrier oil b), liquid under normal conditions, comprises at least one isoparaffinic oil, in particular isodecane, isoundecane, isododecane, isotridecane, isotetradecane, isopentadecane, isohexadecane and isoeicosane.

Further preferred compositions according to the invention are characterized in that the carrier oil b), liquid under normal conditions, comprises a mixture of b)i) a volatile silicone oil, selected from cyclomethicone and linear polydimethylsiloxanes with 2-10 siloxane units, and of b)ii) at least one isoparaffinic oil, isodecane, isoundecane, isododecane, isotridecane, isotetradecane, isopentadecane, isohexadecane and isoeicosane.

In addition to the aforementioned substances usually designated as “volatile” silicone oils, as well as in addition to the aforementioned volatile non-silicone oils, more preferred antiperspirant compositions according to the invention further contain at least one non-volatile cosmetic oil chosen from non-volatile silicone oils and non-volatile non-silicone oils. The at least one non-volatile oil compensates the negative effect of the volatile oil on the residual behavior of preferred antiperspirant compositions according to the invention.

By the relatively fast evaporation of the volatile oils, solid insoluble components, in particular, antiperspirant active substances, may be visible on the skin as an unaesthetic residue. These residues may be successfully masked with a non-volatile oil. Additionally, with a mixture of non-volatile and volatile oil, parameters such as skin sensation, visibility of the residue and stability of the suspension, may be finally adjusted and better adapted to the needs of the consumer.

Of course, it is also possible to formulate anhydrous antiperspirant compositions with a low content of volatile oils, or even without any volatile oils.

Preferred non-volatile silicone oils include high molecular linear dimethylpolysiloxanes, commercially available, for example, under the designation Dow Corning® 190, Dow Corning® 200 Fluid with kinematic viscosities (25° C.) in the range of 5-100 cSt, preferably 6-50 cSt or even 5-10 cSt, and Baysilon® 350 M (with a kinematic viscosity (25° C.) of about 350 cSt). Also preferred silicone oils according to the invention include silicones of formula (Sil-1), wherein x is an integer from 1 to 20.

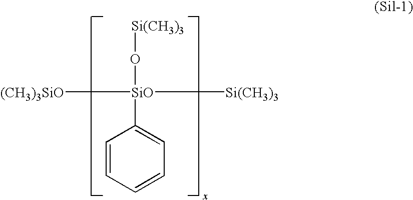

A preferred silicone oil of formula (Sil-1) is available under the INCI designation phenyl trimethicone in various qualities, viscosities and volatilities. A non-volatile phenyl trimethicone is, for example, available from Dow Corning under the designation Dow Corning 556.

Natural and synthetic hydrocarbons, such as paraffin oils, C₁₈-C₃₀ isoparaffins, in particular isoeicosanes, polyisobutenes or polydecenes, which are available for example under the designation Emery° 3004, 3006, 3010 or under the designation Ethyiflo® from Albemarle or Nexbase° 2004G from Nestle, as well as 1,3-di-(2-ethylhexyl)-cyclohexane (available for example under the trade names Cetiol®S from Cognis) also belong to the preferred non-volatile non-silicone oils according to the invention.

Further preferred non-volatile non-silicone oils according to the invention are selected from benzoic acid esters of linear or branched C₈-₂₂ alkanols. Benzoic acids C₁₂-C₁₅ alkylesters are more preferred, for example available as the commercial product Finsoly® TN, benzoic acid isostearyl ester, for example available as the commercial product Finsoly ® SB, ethylhexyl benzoate, for example available as a commercial product Finsoly® EB, and benzoic acid octyldodecyl ester, for example available as a commercial product Finsoly® BOD. Such benzoic acid ester oils are particularly well suitable for masking antiperspirant active substance residues, since their refractive index comes very close to particularly effective aluminum-zirconium mixed salts.

Further preferred non-volatile non-silicone oils according to the invention include branched, saturated or unsaturated fatty alcohols with 6-30 carbon atoms. These alcohols are also often designated as Guerbet alcohols, since they may be obtained from the Guerbet reaction. Preferred alcohol oils are hexyldecanol (Eutanol® G 16, Guerbitol® T 16), octyldodecanol (Eutanol® G, Guerbitol® 20), 2-ethylhexyl alkohol and the commercial products Guerbitol® 18, Isofol® 12, Isofol® 16, Isofol® 24, Isofol® 36, Isocarb® 12, Isocarb® 16 oder Isocarb® 24.

Further preferred non-volatile non-silicone oils include mixtures of Guerbet alcohols and Guerbet alcohol esters, for example, the commercial product Cetiol® PGL (hexyldecanol and hexyldecyl laurate).

Further preferred non-volatile non-silicone oils include triglycerides of linear or branched saturated or unsaturated, optionally hydroxylated C₈₋₃₀ fatty acids. The use of natural oils may be particularly suitable, for example, soya bean oil, cotton seed oil, sunflower oil, palm oil, palm kernel oil, linseed oil, almond oil, castor oil, maize oil, rapeseed oil, olive oil, sesame oil, safflower oil, wheat germ oil, peach stone oil, and the liquid proportions of coconut oils, and the like. However, synthetic triglyceride oils are also suitable, particularly capric/caprylic triglycerides, for example, the commercial products Myritol® 318, Myritol® 331 (Cognis) or Miglyol® 812 (Hülls) with non-branched fatty acid residues as well as glyceryl triisostearin and the commercial products Estol® GTEH 3609 (Uniqema) or Myritol® GTEH (Cognis) with branched fatty acid residues.

Further more preferred non-volatile non-silicone oils include dicarboxylic acid esters of linear or branched C₂-C ₁₀ alkanols, in particular diisopropyl adipate, di-n-butyl adipate, di-(2-ethylhexyl)adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl-succinate and di-(2-hexyldecyl)-succinate.

Further more preferred non-volatile non-silicone oils according to the invention include esters of linear or branched, saturated or non-volatile non-silicone oils, unsaturated fatty alcohols with 6-30 carbon atoms with linear or branched saturated or unsaturated fatty acids with 2-30 carbon atoms, which may be hydroxylated. To these belong isopropyl myristate, isopropyl palmitate, isopropyl isostearate, hexyldecyl stearate (Eutanol® G 16 S), hexyldecyl laurate, isononyl isononanoate, 2-ethylhexyl palmitate (Cegesoft® C 24) and 2-ethylhexyl stearate (Cetiol® 868). Isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid 2-butyloctanoate, diisotridecylacetate, n-hexyl laurate, n-decyl oleate, oleyl oleate, oleyl erucate, erucyl oleate and erucyl erucate are also preferred.

Further more preferred non-volatile non-silicone oils according to the invention include addition products of 1 to 5 propylene oxide units on monohydric or polyhydric C₈₋₂₂ alkanols such as octanol, decanol, decane diol, lauryl alcohol, myristyl alcohol and stearyl alcohol, for example PPG-2-myristyl ether and PPG-3-myristyl ether (Witconol® APM).

Further more preferred non-volatile non-silicone oils according to the invention include addition products of at least 6 ethylene oxide and/or propylene oxide units on monohydric or polyhydric C₃-₂₂ alkanols such as glycerol, butanol, butanediol, myristyl alcohol and stearyl alcohol, which, if desired, may be esterified, for example PPG-14-butylether (Ucon Fluid® AP), PPG-9-butylether (Breox® B25), PPG-10-butane diol (Macol® 57), PPG-15-stearyl ether (Arlamol® E) and glycereth-7-diisononanoate.

Further more preferred non-volatile non-silicone oils according to the invention include symmetrical, unsymmetrical or cyclic esters of carbonic acid with fatty alcohols, for example glycerol carbonate, dicaprylyl carbonate (Cetiol® CC) or the esters according to the teaching of DE 19756454 A1.

Further oils, which may be preferred according to the invention, include esters of dimers of unsaturated C₁₂-C₂₂ fatty acids (dimeric fatty acids) with monohydric linear, branched or cyclic C₂-C₁₈ alkanols or with polyhydric linear or branched C₂-C₆ alkanols.

According to the invention it may be preferred to apply mixtures of the aforementioned oils. Preferred compositions according to the invention include a carrier oil, liquid under normal conditions, chosen from volatile silicone oils, non-volatile silicone oils, volatile hydrocarbon oils, branched, saturated or unsaturated fatty alcohols with 6-30 carbon atoms, triglycerides of linear or branched, saturated or unsaturated, optionally hydroxylated C₈₋₃₀ fatty acids, dicarboxylic acid esters of linear or branched C₂-C₁₀ alkanols, esters of branched, saturated or unsaturated fatty alcohols with 2-30 carbon atoms with linear or branched, saturated or unsaturated fatty acids with 2-30 carbon atoms, which may be hydroxylated, addition products of 1 to 5 propylene oxide units on monohydric or polyhydric C₈₋₂₂ alkanols, addition products of at least 6 ethylene oxide and/or propylene oxide units on monohydric or polyhydric C₃₋₂₂ alkanols, C₈-C₂₂ fatty alcohol esters of monohydric or polyhydric C₂-C₇ hydroxycarboxylic acids, symmetrical, unsymmetrical or cyclic esters of carbonic acid with fatty alcohols, esters of dimers of unsaturated C₁₂-C₂₂ fatty acids (dimeric fatty acids) with monohydric linear, branched or cyclic C₂-C₁₈ alkanols or with polyhydric linear or branched C₂-C₆ alkanols as well as mixtures of the aforementioned substances.

According to the invention it may be most preferable to apply mixtures of the aforementioned oils in order to obtain an optimum fine adjustment of the product properties, in particular of the residue behavior, of the skin sensation or the active substance release.

More preferred compositions according to the invention include those wherein the carrier oil, liquid under normal conditions, is a mixture of at least one volatile silicone oil and at least one oil of a different type. These are preferably mixtures of at least one volatile silicone oil and at least one non-silicone oil, selected from volatile hydrocarbon oils, branched, saturated or unsaturated fatty alcohols with 6-30 carbon atoms, triglycerides of linear or branched, saturated or unsaturated, optionally hydroxylated C₈₋₃₀ fatty acids, dicarboxylic acid esters of linear or branched C₂-C₁₀ alkanols, esters of branched, saturated or unsaturated fatty alcohols with 2-30 carbon atoms with linear or branched, saturated or unsaturated fatty acids with 2-30 carbon atoms, which may be hydroxylated, addition products of 1 to 5 propylene oxide units on monohydric or polyhydric C₈₋₂₂ alkanols, addition products of at least 6 ethylene oxide and/or propylene oxide units on monohydric or polyhydric C₃₋₂₂ alkanols, C₈-C₂₂ fatty alcohol esters of monohydric or polyhydric C₂-C₇ hydroxycarboxylic acids, symmetrical, unsymmetrical or cyclic esters of carbonic acid with fatty alcohols, esters of dimers of unsaturated C₁₂-C₂₂ fatty acids (dimeric fatty acids) with monohydric linear, branched or cyclic C₂-C₁₈ alkanols or with polyhydric linear or branched C₂-C₆ alkanols as well as mixtures of the aforementioned substances.

Further, more preferred oil component types according to the invention are isoparaffin oils, in particular isododecane, isohexadecane and isoeicosane. Isododecane, isohexadecane and isoeicosane belong to the volatile oil components. Since they evaporate relatively rapidly after application on the skin, the hydrophobic load of antiperspirant active substance particles is reduced. Such volatile oil components thus assist with the release of the antiperspirant active substance.

Compositions according to the invention preferably exist as a suspension, meaning, the antiperspirant active substance and optionally further insoluble constituents are suspended in a liquid carrier, which is optionally thickened or solidified into a stick.

Manufacture of compositions according to the invention (which are applied as a non-aerosol) is preferably determined according to the requirements of the type of application. Preferred compositions according to the invention exist in solid, semi-solid, liquid, disperse, anhydrously emulsified, suspended or gelled form.

In a more preferred embodiment, compositions according to the invention exist in liquid form. In the sense of the invention, also any solid dispersions in liquids come under the term of liquid. Compositions according to the invention may also exist as pastes, ointments, lotions or creams. Solid compositions may exist for example as loose powder, pressed powder or a stick.

For example, in the case of liquid agents, the application may also be performed with a roller applicator, as it is known for example from the field of deodorant rollers. Such rollers have a ball mounted in a ball bed, which may be moved by motion over a surface. The ball then takes some of the agent to be distributed and conveys the latter onto the surface to be treated.

Preferred cosmetic sticks according to the invention include carrier oil, liquid under normal conditions, chosen from volatile silicone oils, non-volatile silicone oils, volatile hydrocarbon oils, branched, saturated or unsaturated fatty alcohols with 6-30 carbon atoms, triglycerides of linear or branched, saturated or unsaturated, optionally hydroxylated C₈₋₃₀ fatty acids, dicarboxylic acid esters of linear or branched C₂-C₁₀ alkanols, esters of branched, saturated or unsaturated fatty alcohols with 2-30 carbon atoms with linear or branched or unsaturated fatty acids with 2-30 carbon atoms, which may be hydroxylated, addition products of 1 to 5 propylene oxide units on monohydric or polyhydric C₈₋₂₂ alkanols, addition products of at least 6 ethylene oxide and/or propylene oxide units on monohydric or polyhydric C₃₋₂₂ alkanols, C₈-C₂₂ fatty alcohol esters of monohydric or polyhydric C₂-C₇ hydroxycarboxylic acids, symmetrical, unsymmetrical or cyclic esters of carbonic acid with fatty alcohols, esters of dimers of unsaturated C₁₂-C₂₂ fatty acids (dimeric fatty acids) with monohydric linear, branched or cyclic C₂-C₁₈ alkanols, or with polyhydric linear or branched C₂-C₆ alkanols, as well as mixtures of the aforementioned substances.

It may be most preferred according to the invention to apply mixtures of the aforementioned oils, in order to obtain an optimum fine adjustment of the product properties, in particular of the stick properties, such as stick hardness, residue behavior, abrasion properties or active substance release.

Further preferred compositions according to the invention, in particular those in the form of sticks and soft solids, contain at least one fatty component solid under normal conditions with a melting point above 50-120° C.

Preferred fatty components with a melting point >50-120° C. are chosen from waxes. Generally waxes are of a solid up to brittle hard consistency, coarse to fine crystalline, transparent to opaque, however not glassy, and melt above 50° C. without decomposition. Already a little above the melting point, they are of low viscosity and exhibit strong temperature-dependent consistency and solubility.

For example, natural vegetable waxes are preferred according to the invention such as candelilla wax, carnauba wax, Japan wax, sugarcane wax, ouricoury wax, cork wax, sunflower was, fruit waxes such as orange waxes, lemon waxes, grapefruit wax, and animal waxes, for example, beeswax, shellac wax, and ambergris. In the sense of the invention, it may be more preferred to apply hydrogenated or hardened waxes. As a wax component, chemically modified waxes, in particular the hard waxes, such as for example montan ester waxes, hydrogenated jojoba waxes and sasol waxes may also be applied. To the synthetic waxes, which are also preferred according to the invention, belong for example polyalkylene waxes, in particular polyethylene waxes and polyethyleneglycol waxes, C₂₀-C₄₀ dialkylesters of dimeric acids, C₃₀₋₅₀-alkyl beeswax as well as alkyl- and alkylaryl-esters of dimeric fatty acids.

A more preferred wax component is selected from at least one ester of a saturated, monovalent C₁₆-C₆₀ alcohol and of a saturated C₈-C₃₆ monocarboxylic acid. According to the invention, lactides also belong to them, which are cyclic double esters of a-hydroxycarboxylic acids of corresponding chain length. Esters from fatty acids and long chain alcohols have prove to be particularly advantageous for the preferred compositions according to the invention, because they impart excellent sensorial properties and high stability to the sticks. The esters consist of saturated, branched or non-branched monocarboxylic acids and saturated, branched or non-branched monovalent alcohols. Also esters from aromatic carboxylic acids or hydroxycarboxylic acids (for example 12-hydroxystearic acid) and saturated, branched or non-branched alcohols may be applied according to the invention, insofar that the wax component has a melting point >50° C. It is more preferred to choose wax components from the group of esters from saturated, branched or non-branched alkane-carboxylic acids with a chain length of 12 to 24 C atoms and the saturated, branched or non-branched alcohols with a chain length of 16 to 50 C atoms, which have a melting point >50° C.

In particular as wax components, C₁₆₋₃₆ alkylstearates and C₁₈₋₃₈ alkylhydroxystearoyl stearates, C₂₀₋₄₀ alkyl erucates as well as cetearyl behenate may be preferred. The wax or the wax components have a melting point >50° C., preferably >60° C.

A more preferred embodiment of the invention contains as a wax component, a C₂₀-C₄₀ alkyl stearate. This ester is known under the name of Kesterwachs® K82H or Kesterwachs® K8OH and is marketed by Koster Keunen Inc. This is the synthetic imitation of the monoester fraction of beeswax and is distinguished by its hardness, its oil gelling capacity and its wide compatibility with lipid components. Kesterwachs provides the advantage that it has excellent oil gelling capacity even for low concentrations and thus does not make the stick or soft solid mass too heavy and allows smooth abrasion. A further more preferred embodiment of the invention contains as a wax component cetearyl behenate (i.e., mixtures of cetyl behenate and stearyl-behenate). This ester is known under the name of Kesterwachs® K62 and is marketed by Koster Keunen Inc.

Further preferred wax components with a melting point >50° C. are the triglycerides of saturated and optionally hydroxylated C₁₂₋₃₀ fatty acids, such as hardened triglyceride fats (hydrogenated palm oil, hydrogenated coconut oil, hydrogenated castor oil), glyceryl tribehenate (tribehenin) or glyceryl tri-12-hydroxystearate, further synthetic full esters from fatty acids and glycols or polyols with 2-6 carbon atoms, insofar that they have a melting point above 50° C., for example preferably C₁₈-C₃₆ acid triglyceride (Syncrowax® HGL-C).

According to the invention, hydrogenated castor oil as a wax component, commercially available as the product Cutins® HR, is more preferred.

Further preferred wax components with a melting point >50° C. are saturated linear C₁₄-C₃₆ carboxylic acids, in particular myristic acid, palmitic acid, stearic acid and behenic acid as well a mixtures of these compounds, for example Syncrowax® AW 1C (C₁₈-C₃₆ fatty acids) or Cutina® FS 45 (palmitic and stearic acids).

Preferred compositions according to the invention include the wax component as a constituent of the carrier according to the invention chosen from esters from a saturated, monovalent C₁₆-C₆₀ alkanol and a saturated C₈-C₃₆ monocarboxylic acid, in particular cetyl behenate, stearyl behenate, and C₂₀-C₄₀ alkyl stearate, glycerol triesters of saturated linear C₁₂-C₃₀ carboxylic acids, which may be hydroxylated, candilla wax, carnauba wax, beeswax, saturated linear C₁₄-C₃₆ carboxylic acids as well as mixtures of the aforementioned substances. More preferred wax component mixtures are chosen from mixtures of cetyl behenate, stearyl behenate, hardened castor oil, palmitic acid and stearic acid. Further more preferred wax component mixtures include mixtures of C₂₀-C₄₀ alkyl stearate, hardened castor oil, palmitic acid and stearic acid.

More preferred compositions according to the invention include the wax component as a constituent of the carrier according to the invention chosen from mixtures of esters from a saturated monovalent C₁₆-C₆₀ alkanol and a saturated C₈-C₃₆ monocarboxylic acid, in particular C₂₀-C₄₀ alkyl stearate, glycerol triesters of saturated linear C₁₂-C₃₀ carboxylic acid, which may be hydroxylated, in particular hydrogenated castor oil, and saturated linear C₁₄-C₃₆ carboxylic acids, in particular palmitic acid and stearic acid.

Further compositions according to the invention include the wax component(s) as a constituent of the carrier according to the invention present in an amount of 1-10% by weight, preferably 1.5-8% by weight, particularly preferably 2-6% by weight, and most preferably 3-5% by weight, based on total weight of the composition.

Further preferred compositions according to the invention include at least one wax component with a melting point in the range of 25-50° C., chosen from coconut fatty acid glycerol mono-, di- and triesters, butyrospermum Parkii (Shea Butter) and esters of saturated, monovalent C₈-C₁₈ alcohols with saturated C₁₂-C₁₈ monocarboxylic acids as well as mixtures of these substances. These low melting wax components enable optimization of the consistency of the product and a minimization of the visible residues on skin. Commercial products with the INCI designation cocoglycerides are more preferred, particularly preferably a mixture of C₁₂-C₁₈-mono-, di- and triglycerides, which melt in the range of 30-32° C., as available for example under the trade name Novata® AB from Cognis, as well as the products of the Softisan series (Sasol Germany GmbH) with the INCI designation hydrogenated cocoglycerides, in particular Softisan 100, 133, 134, 138, 142. Further preferred esters of saturated, monovalent C₁₂-C₁₈ alcohols with saturated C₁₂-C₁₈ monovalent carboxylic acids are stearyl laurate, cetearyl stearate (for example Crodamol® CSS), stearyl stearate (for example Estol 3706), cetyl palmitate (for example Cutina® CP, melting point: 46-50° C.) and myristyl myristate (for example Cetiol® MM, melting point: 38-42° C.).

Further preferred compositions according to the invention include at least one wax component with a melting point in the range of 25-50° C. present in an amount of from 0.01 to 10% by weight, preferably 0.5-8% by weight, particularly preferably 1-7.5% by weight and most preferably 1.8-7% by weight, further preferably 1.9, 2.0, 2.1, 2.2, 2.3, 2.4, 2.5, 3, 3.5, 4, 4.5, 5. 5.5, 6 and 6.5% by weight, based on total weight of the composition.

Further preferred compositions according to the invention contain, in order to improve consistency and the sensorial properties and in addition to the silicic acid or silicate filler, which is a constituent of the active substance capsules according to the invention, even more or at least one further water-insoluble filler in particle form. In a most preferred embodiment, this filler is selected from optionally modified starches (for example from maize, rice, potatoes) and starch derivatives, which is if desired pre-pasted, silicon dioxide, silicic acids, for example of the Aerosil® type, spherical polyalkylsesquisiloxane particles (in particular Aerosil® R972 and Aerosil® 200V of Degussa), silica gels, talc, kaolin, magnesium-aluminum silicates, boron nitride, lactoglobulin derivatives, for example sodium C₈₋₁₆ iso-alkylsuccinyllactoglobulin sulfonate, available from Brooks Industries as the commercial product Biopol® OE, glass powders, polymer powders, in particular of polyolefins, polycarbonates, polyurethanes, polyamides, for example nylon, polyesters, polystyrenes polyacrylates, (meth)acrylates or (meth)acrylate-vinylidene copolymers, which may be cross-linked, or silicones, as well as mixtures of these substances.

Polymer powder based on a polymethacrylate copolymer are for example available as the commercial product Polytrap® 6603 (Dow Corning). Other polymer powders, for example based on polyamides, are available under the designation Orgasol® 1002 (polyamide-6) and Orgasol® 2002 (polyamide-12) from Elf Atochem. Further polymer powders, which are suitable as preferred fillers according to the invention, are for example polymethacrylates (Micropearl® M from SEPPIC or Plastic Powder A from NIKKOL), styrene-divinylbenzene copolymers (Plastic Powder FP von NIKKOL), polyethylene- and polypropylene powder (ACCUREL® EP 400 from AKZO) or also silicone polymers (Silicone Powder X2-1605 von Dow Corning).

Preferred compositions according to the invention contain at least one solid water-insoluble particulate filler in an amount of from 1 to 99% by weight, preferably 2-90% by weight, particularly preferably 3-15% by weight, most preferably 4, 5, 6, 7, 8, 9, 10, 11, 12, 13 and 14% by weight, based on total composition.

The application may also take place with substrates, onto which a preparation according to the invention is applied. Moist cloths (i.e., pre-fabricated moist cloths for the user), preferably individually packed, as are well-known, for example, from the field of glass cleaning (spectacle cleaning cloth) or from the field of moist toilet tissues. Such moist cloths, which may also preferably contain preservatives, are then impregnated or applied with an agent according to the invention. They may be applied, for example, as antiperspirant tissues, which is of particular interest for a use on the move. It may be more preferred if these cloths are individually packed.

Preferred substrate materials are selected from sheet cloths. They may consist of a fibrous or cellular flexible material which has sufficient mechanical stability and at the same time softness for application on the skin. To these cloths, belong cloths made out of woven or non-woven synthetic and natural fibers, felt, paper or expanded material, such as hydrophilic polyurethane foam.

Preferably, conventional tissues of non-woven material (fleece) are used here. Non-woven fabrics are generally defined as adhesively bonded fiber products, which have matting or a layered fiber structure, or those which comprise fiber mats in which the fibers are randomly distributed or in a statistical arrangement. The fibers may be natural, such as cellulose, lyocell, wool, silk, jute, hemp, cotton, flax, sisal, or ramie; or synthetic, such as rayon, cellulose ester, polyvinyl derivatives, polyolefins, polyamides or polyesters. Generally, each fiber diameter or titer is suitable for the present invention. The non-woven materials applied here because of the random or statistical arrangement of the fibers in the non-woven material, which impart excellent strength in all directions, do not tend to tear or disaggregate. Examples of non-woven materials, which are suitable as substrates in the present invention are known for example from WO 98/18441. Preferred porous and sheet cleaning tissues consist of one or different fiber materials, in particular made of cotton, finished cotton, polyamide, polyester or mixtures thereof. Preferably the substrates in tissue form have a surface from 10 to 5,000 cm², preferably from 50 to 2,000 cm², in particular from 100 to 1,500 cm² and particularly preferably from 200 to 1,000 cm². The surface weight or basis weight of the material is then usually comprised between 20 and 1,000 g/m², preferably from 30 to 500 g/m² and in particular from 50 to 150 g/m². Preferred deodorant substrates according to the invention may be obtained by soaking or impregnation or also by melting the composition according to the invention on a substrate.

Agents according to the invention, preferably liquid agents, may also be multiphase, the phases may be arranged for example horizontally, therefore over each other, or vertically, therefore beside each other. This may also be a disperse system wherein the solid components are distributed inhomogeneously in the liquid matrix, so that such a disperse system should be shaken before their application.

Antiperspirant sticks on an anhydrous basis may exist in a gelled form, the oil phase containing at least one silicone component or may consist of at least one silicone component. Furthermore, compositions according to the invention formulated as antiperspirant sticks may exist on an anhydrous fatty basis, on the basis of a polyol-in-oil emulsion, on the basis of an oil-in-polyol emulsion, on the basis of a polyol-oil multiple emulsion, on the basis of a nano-emulsion and on the basis of a micro-emulsion, the polyol phase being anhydrous. Gel sticks may be formulated on the basis of alditols, in particular dibenzylidene-sorbitol, N-acylamino acid amides, 12-hydroxystearic acid, polyamides and polyamide derivatives.

Anhydrous antiperspirant wax sticks contain approximately 30-70% by weight of at least one cosmetic oil, liquid under normal conditions, approximately 15-25% by weight of a fatty component solid under normal conditions, whereof usually the largest proportion has a melting point of about 50° C. or usually consists of fatty alcohols, in particular stearyl alcohol but also cetyl alcohol, and optionally further arachidyl alcohol and/or behenyl alcohol, while a smaller proportion—approximately 0.5-5% by weight—consists of at least one fatty component with a melting point of about 55-120° C. Furthermore, 0.5-8% by weight of at least one fatty component with a melting point of about 25-35° C. may be contained. Furthermore, 0.5-30% by weight of at least one filler may be contained, which is typically selected from talc, cellulose powders, starches, and starch derivatives. Furthermore, 0.1-10% by weight, preferably 1-5% by weight, particularly preferably 2-4% by weight of at least one non-silicone based oil-in-water emulsifier may be contained.

In a more preferred embodiment according to the invention, the anhydrous antiperspirant wax stick exists as a so-called multiphase stick, in particular as a biphasic stick. Hereunder, sticks according to the invention are meant which contain, for example, a first wax stick phase as a core, and at least one second wax stick phase as a ring around the first phase. In addition to a concentric, ring-shaped arrangement of the different phases, other arrangements are also possible, in particular an arrangement in strip form. The different phases may for example differ from each other by a different coloration, but also by different components. Corresponding multiphase sticks are for example disclosed in U.S. Pat. No. 6,936,242 and WO 00/67712 A1. Preferred preparation methods for such sticks are disclosed in U.S. Pat. No. 6,838,032.

An embodiment as a multiphase stick, in particular as a biphasic stick, in which only one of the phases contains a determined active substance, is more preferred.

A preferred embodiment according to the invention, as an anhydrous antiperspirant wax stick, comprises:
5-40% by weight, preferably 10-35% by weight, particularly preferably 11-28% by weight and most preferably 12-20% by weight (USP) of at least one antiperspirant active substance,
30-70% by weight of at least one cosmetic oil liquid under normal conditions,
15-32% by weight of a fatty component solid under normal conditions, whereof more 65% by weight, preferably more than 70% by weight, particularly preferably more than 80% by weight, each time based on the total content of fatty components solid under normal conditions, from C₁₆-C₃₀ fatty alcohols, preferably selected from the group consisting of stearyl alcohol, cetyl alcohol, and mixtures thereof, wherein particularly preferably in addition to stearyl alcohol and/or cetyl alcohol, further 0.1-3% by weight, based on the total stick, arachidyl alcohol and/or behenyl alcohol and/or at least one C₂₄-C₃₀ fatty alcohol are contained,
furthermore 0.5-5% by weight, based on the total stick, at least one fatty component with a melting point of 75-120° C. and 0.5-8% by weight, based on the total stick, of at least one fatty component with a melting point of 25-45° C.,
further 0.5-30% by weight, preferably 1-25% by weight, particularly preferably 5-20% by weight, most preferably 10-15% by weight of at least one filler, which is particularly preferably chosen from talc, cellulose powders, starches and starch derivatives.

All quantitative indications, if not specified otherwise, refer to the weight of the compositions according to the invention.

### Fragrances

Further preferred compositions according to the invention contain at least one fragrance. As fragrances or perfume oils, different odorous substance compounds may be used, for example the synthetic products of the type: esters, ethers, aldehydes, ketones, alcohols, and hydrocarbons. For example Carvacrol belongs to the phenolic odorous compounds. Odorous substance compounds of the ester type are for example benzyl acetate, methyl anthranilate, ortho-t-butylcyclohexyl acetated, p-tert.-butylcyclohexyl acetate, diethyl phthalate, nonanediol-1,3-di-acetate, iso-nonyl acetate, iso-nonyl formate, phenylethylphenyl acetate, phenoxyethyl isobutyrate, linalyl acetate, dimethylbenzylcarbinyl acetate, phenylethyl acetate, linalyl benzoate, benzyl formate, ethylmethylphenyl glycinate, allylcyclohexyl propionate, styrallyl propionate, benzyl salicylate, ethyl salicylate, iso-amyl salicylate, hexyl salicylate and 4-nonanolide. For example benzylethyl ethers belong to the ethers, linear alkanals with 8 to 18 C atoms, citral, nitronellal, citronellyoxyacetaldehyde, cyclamenaldehyde, hydroxycitronellal, lilial and bourgeonal, belong for example to the aldehydes, for example 6-acetyl-1,1,3,4,4,6-hexamethyl-tetrahydronaphthaline, para-t-amylcyclohexanone, 2-n-heptylcyclopentanone, β-methyl-naphthylketone and the ionones, α-isomethylionone and methylcedrylketone, belong to the ketones, cinnamic alcohol, anethol, citronellol, dimyrcetol, eugenol, geraniol, linalool, phenyl-ethyl alkohol and terpineol, belong to the alcohols, 1,3,4,6,7,8-hexahydro-4,6,6,7,8,8-hexamethylcyclopenta-a-2-benzopyrane, hydroxymethylisopropylcyclopentane, 3-a-methyldodecahydro-6,6,9a-trimethylnaphtho-2(2,1-b)furane, iso-butylquinoline as well as terpenes and balsams belong to the hydrocarbons. Preferably mixtures of different fragrances are used, which produce together a pleasing fragrance note.

Suitable perfume oils may also contain natural odorous substance mixtures accessible from plant or animal sources, for example, pine, citrus, jasmine, rose, lily or ylang-ylang oil. Also etherated oils of lower volatility, which are mostly used as aroma components, are suitable as perfume oils, for example sage oil, chamomile oil, lemon balm oil, mint oil, cinnamon leaf oil, lime blossom oil, juniper berry oil, vetiver oil, olibanum oil, galbanum oil, laudanum oil, clove oil, iso-eugenol, thymian oil, bergamot oil, geranium oil and rose oil. Preferred compositions according to the invention contain at least one fragrance in an amount of 0.1-10% by weight, preferably 0.2-5% by weight, particularly preferably 0.4-4% by weight, most preferably 1-3% by weight, further most preferably 1.5-2.5% by weight, based on total weight of the composition.

Further preferred compositions according to the invention contain at least one so-called “skin-cooling active substance”. Skin-cooling active substances in the sense of the present application mean active substances, which upon application on the skin as a result of surface anaesthetization and stimulation of the cold-sensitive nerves in the case of migraines and the like, produce a pleasant cold feeling, also if the treated skin parts actually exhibit a normal or increased temperature.

Preferred skin-cooling active substances are in particular menthol, isopulegol, as well as menthol derivatives, for example, menthyl lactate, menthylpyrrolidone carboxylic acid, menthyl methylether, menthoxypropane diol, menthane glycerol acetal (9-methyl-6-(1-methylethyl)-1,4-dioxaspiro(4.5)decane-2-methanol), monomenthyl succinate and 2-hydroxymethyl-3,5,5-trimethylcyclohexanol. Menthol, isopulegol, menthyl lactate, menthoxypropane diol and menthylpyrrolidone carboxylic acid are more preferred as skin-cooling active substances. Preferred compositions according to the invention contain at least one skin-cooling active substance in an amount of 0.01-1% by weight, preferably 0.02-0.5% by weight and particularly preferably 0.05-0.2% by weight, based on total weight of the composition.

Preferred compositions according to the invention include at least one encapsulated active substance. Active substances which may advantageously be encapsulated are in particular fragrances, perfume oils and/or skin-cooling active substances, but also other skin care active substances such as vitamins, antioxidants, etc.

Water-soluble polymers such as starch, physically and/or chemically modified starches, cellulose derivatives, such as for example carboxymethylcellulose, methylcellulose, hydroxyethylcellulose or hydroxypropylmethylcellulose, carragheens, alginates, maltodextrines, dextrines, vegetable gums, pectins, xanthans, polyvinyl acetate and polyvinyl alcohol, polyvinylpyrrolidine, polyamides, polyesters and homo- and co-polymers of monomers, selected from acrylic acid, methacrylic acid, maleic acid, fumaric acid, itaconic acid as well as the esters and salts of these acids, as well as any mixtures of these polymers, are preferred as a capsule material.

Preferred capsule materials are chemically modified starches, in particular aluminum starch-octenyl succinate, for example the commercial product Dry Flo Plus from National Starch, or sodium-starch-octenyl succinate, for example the commercial product Capsule from National Starch, further carboxymethyl cellulose, carboxymethyl cellulose, methylcellulose, hydroxyethylcellulose and hydroxypropylmethylcellulose, ethyl cellulose, for example the commercial product Tylose H 10 from Clariant, further carragheens, alginates and maltodextrins, as well as any mixtures of these polymers.

In a further preferred embodiment according to the invention, the compositions according tot he invention contain 0 to a maximal of 5% by weight of ethanol.

The compositions according to the invention are essentially anhydrous, i.e. they contain 0 to at most 3% by weight, preferably 0 to at most 2% by weight of free water, based on the total composition. The content of crystal water, hydration water or similarly molecularly bound water, which may be contained in the applied components, in particular in the antiperspirant active substances, does not represent free water in the sense of the present application.

Furthermore the compositions according to the invention may contain additional deodorants. Anti-microbial, anti-bacterial, or germ inhibiting substances, antioxidants, or odour adsorbers (for example zinc ricinoleate) may be applied as deodorants.

Suitable anti-microbial, anti-bacterial or germ-inhibiting substances are in particular organic halogen compounds as well as organic halides, quaternary ammonium compounds, a series of plant extracts and zinc compounds. Preferred are halogenated phenol derivatives such as for example hexachlorophene or Irgasan DP 300 (Triclosan, 2,4,4′-trichloro-2′-hydroxydiphenylether), 3,4,4′-trichlorocarbonilide, chlorhexidine (1,1′-hexamethylene-bis-[5-(4-chlorphenyl)]-biguanide), chlorhexidine gluconate, benzalkonium halides and cetylpyridinium chloride. Further sodium bicarbonate, sodium phenol sulfonate and zinc phenol sulfonate may be applied as well as for example the constituents of lime blossom oil. Also weaker effective anti-microbial substances, which however have a specific action against Gram-positive germs responsible for the decomposition of sweat, may be applied as deodorant active substances. Also benzyl alcohol may be applied as a deodorant active substance. Further anti-bacterial effective deodorants are lantibiotics, glycoglycerolipids, sphingolipids (ceramids), sterols and other active substances, which inhibit bacteria adhesion on the skin, for example glycosidases, lipases, proteases, carbohydrates, di- and oligo-saccharide fatty acid esters as well as alkylated mono- and oligo-saccharides. Preferred deodorant active substances are long-chain diols, for example 1,2-alkane-(C₈-C₁₅)-diols, glycerol mono(C₈-C₁₈) fatty acid esters, or particularly preferably, glycerol mono(C₆-C₁₆) alkyl ethers, in particular 2-ethylhexyl glycerol ethers, which are very well compatible with skin and mucosae and effective against corynebacteria.

Also complex-forming substances may assist with the deodorant action, by complexing in a stable way the oxidative catalytically acting heavy metal ions (for example iron or copper). Suitable complexing agents are for example the salts of ethylene-diamine tetraacetic acid, or nitrilotriacetic acid as well as the salts of 1-hydroxyethane-1,1-diphosphonic acid.

A further object of the present application is the use of an organosiloxane-oxyalkylene copolymer in an antiperspirant composition, containing at least one antiperspirant active substance and 0-3% by weight, preferably 0-2% by weight of free water for improving reduction of sweat.

By improvement of “sweat reduction”, should be understood according to the invention both a reduction of the amount of sweat and also an acceleration of the release of the antiperspirant active substance from the composition according to the invention.

A further object of the present application is the use of an organosiloxane-oxyalkylene copolymer in an antiperspirant composition, containing at least one antiperspirant active substance and 0-3% by weight, preferably 0-2% by weight of water, for improving sweat reduction, wherein the organosiloxane-oxyalkylene copolymer exists in a composition according to any of claims 1 to 18.

A further object of the present application is the non-therapeutic, cosmetic use of an antiperspirant composition according to claim 1, 2, 3, 4, 5, 6, 7, 8, 9, 10, 11, 12, 13, 14, 15, 16, 17 or 18 for reducing and/or regulating perspiration and/or body odour.

As regards further preferred embodiments of the uses according to the invention, what was stated concerning the compositions according to the invention applies mutatis mutandis.

A further object of the present application is a non-therapeutic, cosmetic method for reducing and/or regulating sweat formation and/or body odour, in which a composition according to the invention or preferred according to the invention, according to any of claims 1, 2, 3, 4, 5, 6, 7, 8, 9, 10, 11, 12, 13, 14, 15, 16, 17 or 18, is applied in an effective amount on the skin, preferably on the skin in the armpit area.

As regards further preferred embodiments of the method according to the invention, what was said concerning the compositions according to the invention applies mutatis mutandis.

The following examples should explicit the invention without it being limited thereto.

Antiperspirant suspension compositions for making as a roll-on in a roll-ball dispenser (all weight indications are % by weight).

| Commercial name | INCI | No. 1 | No. 2 | No. 3 | No. 4 | No. 5 | No. 6 |
|---|---|---|---|---|---|---|---|
| | Activated Aluminum | 22.0 | 22.0 | 22.0 | 22.0 | — | — |
| | Zirconium Pentachloro- | | | | | | |
| | hydrex - Gly | | | | | | |
| Reach AZP 908 | Activated Aluminum | — | — | — | — | 24 | 24 |
| | Zirconium Tetrachloro- | | | | | | |
| | hydrex - Gly | | | | | | |
| Dow Corning 245 | Cyclopentasiloxane | 70.6 | 0.6 | 70.6 | 70.6 | — | — |
| Fluid | | | | | | | |
| Dow Corning 345 | Cyclopentasiloxane/ | — | — | — | — | 66.8 | 64.8 |
| Fluid | Cyclohexasiloxane | | | | | | |
| Mirasil DMCO | PEG/PPG-22/24 | 2 | — | — | — | — | 2 |
| | dimethicone | | | | | | |
| Dow Corning 193 | PEG-12 dimethicone | — | 2 | — | — | — | — |
| Abil B 88184 | PEG/PPG-20/6 | — | — | 2 | — | — | — |
| | dimethicone | | | | | | |
| Abil B 8851 | PEG/PPG-14/4 | — | — | — | 2 | — | — |
| | dimethicone | | | | | | |
| Abil EM 97 | Bis-PEG/PPG-14/14 | — | — | — | — | 2 | — |
| | dimethicone | | | | | | |
| Abil B 8832 | Bis PEG/PPG-20/20 | — | — | — | — | — | 2 |
| | dimethicone | | | | | | |
| Dow Corning | PEG/PPG-17/18 | — | — | — | — | — | — |
| Q2-5220 | dimethicone | | | | | | |
| Propylene | Propylene Carbonate | 0.9 | 0.9 | 0.9 | 0.9 | 1.2 | 1.2 |
| carbonate | | | | | | | |
| Bentone 38 V CG | Disteardimonium | 3.8 | 3.8 | 3.8 | 3.8 | 5 | 5 |
| | Hectorite | | | | | | |
| Parfum InCaps | Incapsulated Fragrance | 0.2 | 0.2 | 0.2 | 0.2 | — | — |
| Parfum | Fragrance | 0.5 | 0.5 | 0.5 | 0.5 | 1 | 1 |
| Commercial name | INCI | No. 7 | No. 8 | No. 9 | No. 10 | No. 11 | No. 12 |
| | Activated Aluminum | — | — | 22 | 22 | 22 | 22 |
| | Zirconium Pentachloro- | | | | | | |
| | hydrex - Gly | | | | | | |
| Reach AZP 908 | Activated Aluminum | 24 | 24 | — | — | — | — |
| | Zirconium Tetrachloro- | | | | | | |
| | hydrex - Gly | | | | | | |
| Dow Corning 245 | Cyclopentasiloxane | — | — | 66.8 | 65.8 | 66.8 | 65.8 |
| Fluid | | | | | | | |
| Dow Corning 345 | Cyclopentasiloxane/ | 66.8 | 66.8 | — | — | — | — |
| Fluid | Cyclohexasiloxane | | | | | | |
| Mirasil DMCO | PEG/PPG-22/24 | — | — | 2 | 2 | 2 | 2 |
| | dimethicone | | | | | | |
| Dow Corning 193 | PEG-12 dimethicone | — | — | 2 | — | — | — |
| Abil B 88184 | PEG/PPG-20/6 | — | — | — | 2 | — | — |
| | dimethicone | | | | | | |
| Abil B 8851 | PEG/PPG-14/4 | — | — | — | — | 2 | — |
| | dimethicone | | | | | | |
| Abil EM 97 | Bis-PEG/PPG-14/14 | — | — | — | — | — | — |
| | dimethicone | | | | | | |
| Abil B 8832 | Bis PEG/PPG-20/20 | 2 | — | — | — | — | — |
| | dimethicone | | | | | | |
| Dow Corning | PEG/PPG-17/18 | — | 2 | — | — | — | 2 |
| Q2-5220 | dimethicone | | | | | | |
| Propylene | Propylene Carbonate | 1.2 | 1.2 | 1.2 | 1.2 | 1.2 | 1.2 |
| carbonate | | | | | | | |
| Bentone 38 V CG | Disteardimonium | 5 | 5 | 5 | 5 | 5 | 5 |
| | Hectorite | | | | | | |
| Parfum InCaps | Encapsulated Fragrance | — | — | — | 1 | — | 1 |
| Parfum | Fragrance | 1 | 1 | 1 | 1 | 1 | 1 |

Measurement of the release of the antiperspirant active substance

In order to demonstrate that the antiperspirant active substance is rapidly available, the time course of the conductivity of the suspensions from a defined film is measured in a determined amount of deionized water.

The value for the conductivity attained at the end of the test for the compositions according to the invention is around 80-160 microsiemens [μS] per centimeter.

The compositions of the state of the art have a final conductivity of maximum 10-50 microsiemens per centimeter.

Compositions according to the invention Nos. 1-12 were applied on the skin in the armpit area.

We claim:

1. Antiperspirant composition for personal body care, ready-made as a non-aerosol, a stick, a soft solid, a cream, a gel, a suspension, a solution or impregnated on a substrate, comprising:
a) at least one antiperspirant active substance,
b) at least one oil, liquid under normal conditions, as a carrier,
c) 0-3 wt. % by weight of free water, based on total weight of the composition, and
d) at least one organosiloxane-oxyalkylene copolymer.

2. Composition according to claim 1, wherein the at least one organosiloxane-oxyalkylene copolymer is chosen from compounds of general structural formulae (I), (II), (III), (IV) and (V) 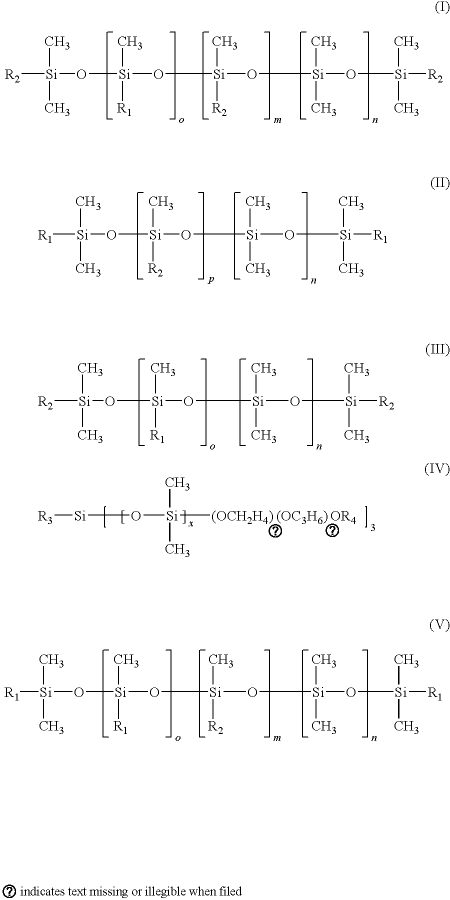
wherein
R¹ is independently a linear or branched C₁-C₃₀ alkyl group, or an optional substituted phenyl group,
R² is —CH_{c}H_{2c}—O—(C₂H₄O—)ₐ(C₃H₆O—)_{b}R⁵ or —C_{c}H_{2c}—O—(CH₂H₄O—)ₐR⁵ or —CH₂—CH(CH₃)—CH₂—O—(C₂H₄O—)ₐ(C₃H₆O—)_{b}R⁵,
R³ and R⁴ are independently a linear or branched C₁-C₁₆ alkyl group,
R⁵ is a hydrogen atom or a methyl group,
m is a number from 0-20,
n is a number from 0-500,
o is a number from 0-20,
p is a number from 1-50,
a is a number from 0-50,
b is a number from 0-50,
a+b has the value of at least 1,
c is a number from 1-4, and
x is a number from 1-100.

3. Composition according to claim 1, wherein the at least one organosiloxane-oxyalkylene copolymer is chosen from linear polysiloxane-polyoxyalkylene block copolymers.

4. Composition according to claim 1, wherein the at least one organosiloxane-oxyalkylene copolymer has an HLB value of from 8 to 20.

5. Composition according to claim 2, wherein the at least one organosiloxane-oxyalkylene copolymer is at least compounds according to general structural formula (II).

6. Composition according to claim 1, wherein the organosiloxane-oxyalkylene copolymer is an organosiloxane-oxyalkylene copolymer having an average number of silicon atoms per molecule of 15 or less.

7. Composition according to claim 2, wherein at least one organosiloxane-oxyalkylene copolymer is at least one organosiloxane-oxyalkylene copolymer of general structural formula (II) comprising an HLB value of 8-20, wherein
R¹=methyl,
R²═—(CH₂)₃—O—(C₂H₄O—)₂₂ (C₃H₆O—)₂₄CH₃
n=10-500, and
p=10-50.

8. Composition according to claim 2, wherein at least one organosiloxane-oxyalkylene copolymer is at least one organosiloxane-oxyalkylene copolymer of general structural formula (II) comprising an HLB value of 8-20, wherein
R¹=methyl group,
R²═—C_{c}H_{2c}—O—(C₂H₄O—)ₐR⁵,
with
a=5-20,
b=0,
c=3, and
R⁵=a hydrogen atom or a methyl group,
n=0, and
p=1.

9. Composition according to claim 2, wherein at least one organosiloxane-oxyalkylene copolymer is at least one organosiloxane-oxyalkylene copolymer of general structural formula (II) comprising an HLB value of 8-20, wherein
R¹=tert.-butyl group,
R²═—C_{c}H_{2c}—O—(C₂H₄O—)ₐR⁵,
with
a=5-20,
b=0,
c=3, and
R⁵=a hydrogen atom or a methyl group,
n=0, and
p=1.

10. Composition according to claim 2, wherein at least one organosiloxane-oxyalkylene copolymer is at least one organosiloxane-oxyalkylene copolymer of general structural formula (II) comprising an HLB value of 8-20, wherein
R¹=isopropyl group,
R²═—C_{c}H_{2c}—O—(C₂H₄O—)ₐR⁵,
with
a=5-20,
c=3, and
R⁵=a hydrogen atom or a methyl group,
n=0, and
p=1.

11. Composition according to claim 2, wherein at least one organosiloxane-oxyalkylene copolymer is at least one organosiloxane-oxyalkylene copolymer of general structural formula (II) comprising an HLB value of 8-20, wherein
R¹=methyl,
R²═—CH₂—CH(CH₃)—CH₂—O—(C₂H₄O—)ₐR⁵, with
a=5-20,
c=3, and
R⁵=a hydrogen atom or a methyl group,
n=0, and
p=1.

12. Composition according to claim 1, wherein the at least one organosiloxane-oxyalkylene copolymer is at least a linear polysiloxane-polyoxyethylene-polyoxypropylene block copolymer having 1NCI designation PEG/PPG-22/24 dimethicone.

13. Composition according to claim 1, wherein the at least one organosiloxane-oxyalkylene copolymer has a water solubility of at least 5 g per 100 g of aqueous solution.

14. Composition according to claim 1 further comprising at least one lipophilic thickener.

15. Composition according to claim 1, wherein the at least one oil carrier which is liquid under normal conditions is chosen from volatile cyclic or linear silicone oils, non-volatile higher molecular linear dimethylpolysiloxanes, esters of linear or branched, saturated or unsaturated fatty alcohols with 2-30 carbon atoms, linear or branched, saturated or unsaturated, optionally hydroxylated, fatty acids with 2-30 carbon atoms, benzoic acid esters of linear or branched C₈₋₂₂ alkanols, C₈-C₂₂ fatty alcohol esters of monohydric or polyhydric C₂-C₇ hydroxycarboxylic acids, addition products of ethylene oxide and/or propylene oxide on monohydric or polyhydric C₃₋₂₀ alkanols, liquid paraffin oils, isoparaffin oils, in particular isodecane, isoundecane, isododecane, isotridecane, isotetradecane, isopentadecane, isohexadecane and isoeicosane, synthetic hydrocarbons, such as polyisobutene or polydecenes, and alicyclic hydrocarbons, branched saturated or unsaturated fatty alcohols with 6-30 carbon atoms, mixtures of Guerbet alcohols and Guerbet alcohol esters, symmetrical, unsymmetrical or cyclic esters of carbonic acid with fatty alcohols, triglycerides of linear or branched, saturated or unsaturated, optionally hydroxylated C₈₋₃₀ fatty acids, dicarboxylic acid esters of linear or branched C₆-C₁₀ alkanols, di-n-alkylethers having 12 to 36C atoms, as well as mixtures thereof.

16. Composition according to claim 1, wherein the at least one oil carrier which is liquid under normal conditions is at least isoparaffin oil.

17. Composition according to claim 1, wherein the at least one oil carrier which is liquid under normal conditions is at least a mixture of volatile silicone oil chosen from cyclomethicone and linear polydimethylsiloxanes with 2-10 siloxane units and at least one isoparaffin oil.

18. Composition according to claim 1, wherein the at least one oil carrier which is liquid under normal conditions is at least a mixture of volatile silicone oil and at least one non-silicone oil chosen from branched, saturated or unsaturated fatty alcohols with 6-30 carbon atoms, triglycerides of linear or branched, saturated or unsaturated, optionally hydroxylated, C₈₋₃₀ fatty acids, dicarboxylic acid esters of linear or branched C₂-C₁₀ alkanols, esters of branched, saturated or unsaturated fatty alcohols with 2-30 carbon atoms with linear or branched, saturated or unsaturated fatty acids with 2-30 carbon atoms, optionally hydroxylated, addition products of 1 to 5 propylene oxide units on monohydric or polyhydric C₈₋₂₂ alkanols, addition products of at least 6 ethylene oxide and/or propylene oxide units on monohydric or polyhydric C₃₋₂₂ alkanols, C₈-C₂₂ fatty alcohol esters of monohydric or polyhydric C₂-C₇ hydroxycarboxylic acids, symmetrical, unsymmetrical or cyclic esters of carbonic acid with fatty alcohols, and esters of dimers of unsaturated C₁₂-C₂₂ fatty acids with monohydric linear, branched or cyclic C₂-C₁₈ alkanols or with polyhydric linear or branched C₂-C₆ alkanols.

19. Non-therapeutic cosmetic method for reducing and/or regulating formation of sweat and/or of body odor comprising:
applying an effective amount of a composition according to claim 1 onto skin.
